(19) United States
(12) Patent Application Publication
(10) Pub. No.: US 2008/0261226 A1
(43) Pub. Date: Oct. 23, 2008

(54) Biomarkers of neurodegenerative disease

(76) Inventors: Rengang Wang, San Diego, CA (US); Dongxian Zhang, La Jolla, CA (US)

Correspondence Address: Biotechnology Law Group;c/o Portfolioip, P.O. Box 52050, Minneapolis, MN, 55402, US

(21) Appl. No.: 12/070,218

(22) Filed: Feb. 15, 2008

Related U.S. Application Data
(60) Provisional application No. 60/901,721, filed on Feb. 15, 2007.

(51) Int. Cl. C12Q 1/68; G01N 33/53; C12Q 1/02

(52) U.S. Cl. 435/6; 435/7.1; 435/7.92; 435/29

(57) ABSTRACT

The present invention provides biomarkers and diagnostic methods employing such biomarkers based on the discovery of genes that have a two-fold or greater difference in gene expression in the spinal cord of a pre-symptomatic mouse model of amyotrophic lateral sclerosis. Such biomarkers and diagnostic methods are useful for early detection of neural cell injury and death in acute and degenerative disease.

### STATEMENT OF GOVERNMENT RIGHTS

The invention was supported, at least in part, by a grant from the Government of the United States of America (grant no. R21 DE015129-01 A1 from the National Institutes of Health (NIH/DEO). The Government may have certain rights to the invention.

### FIELD OF THE INVENTION

The present invention relates to the field of diagnostics for neurodegenerative disease. In particular, the present invention relates to biomarkers for early detection of neurodegenerative disease.

### BACKGROUND OF THE INVENTION

### Biomarkers of Neurodegenerative Disease

Novel biomarkers of neurodegenerative disease are required for both diagnosis and treatment. Neural damage occurs prior to the onset of clinical symptoms of disease (Edelman and Gally, Proc. Nat. Acad. Sci. USA 98:13763-13768, 2001; Prinz et al., Nat. Neurosci. 7:1345-1352, 2004). Molecular and cellular events that occur after the onset of clinical symptoms may reflect late stages of dying cells, rendering treatment based on the detection of these events largely ineffective. Therefore, biomarkers identifying early disease events permit the effective diagnosis and treatment of neurodegenerative disease. Additionally, clinical symptoms of neurodegenerative disease are often complex and vague, providing limited information as to the scope and severity of damage to affected areas of the brain. Moreover, these diseases reflect damage to many connected areas of the brain. As a result, clinical symptoms alone do not define the role of a specific area of the brain in disease progression. Biomarkers that can reveal the location and severity of neural injury will assist tremendously in monitoring disease progression. Finally, protocols for evaluating clinical symptoms or behavior in animal models vary, and the results obtained are difficult to compare among individual cases. Assays based on biomarkers will help standardize tests and provide comparable results, enabling effective diagnosis and treatment.

Clinical biomarkers can be used to diagnose diseases and monitor their progression, select treatments and compare the results of various treatment options. Biomarkers are also useful in basic research on animal models of neurodegenerative disease. Biomarkers also facilitate the study of early molecular events in a disease process and the identification of targets for intervention. Knowledge of pre-symptomatic events may lead to prevention, which is likely the most effective strategy against these diseases. Therefore, the use of biomarkers should not only facilitate the early diagnosis of neurodegenerative diseases, but may also lead to more effective strategies for disease prevention, treatment, and ultimately cure.

Ideal biomarkers of neurodegenerative disease have the following characteristics. First, they reflect the health of neural cells. Because most diseases are sporadic and affect specific groups of cells, monitoring the viability of neural cells is far more important than identifying their genetic traits. Second, such biomarkers show reproducibility and specificity. As disease manifestation is often influenced by genetic and epigenetic factors, effective biomarkers reliably identify specific cells that have been injured by a disease and be expressed in all affected individuals. Third, they are highly sensitive and robust to permit early detection and localization of neurodegenerative diseases, which often advance gradually, affecting only a few neural cells in early stages. Finally, such biomarkers are detectable by noninvasive methods such as in vivo imaging or examination of body fluids.

Amyotrophic lateral sclerosis (ALS) is one example of a neurodegenerative for which useful biomarkers are lacking. ALS is an adult-onset neurodegenerative disease characterized by the loss of specific motor neurons in the spinal cord, brainstem and cortex (Borchelt et al., Brain Pathology 8:735-757, 1998; Cleveland, Neuron 24:515-520, 1999; Cole and Siddique, Seminars in Neurology 19:407-418, 1999; Haverkamp et al., Brain 118:707-719, 1995; Munsat et al., Neurology 38:409-413, 1988; Rowland, Adv. Neurology 36:1-13, 1982; Rowland and Schneider, New England J. Med. 344:1688-1700, 2001; Wong et al., Curr. Opin. Neurobiol. 8:791-799, 1998). ALS affects approximately 5 in 100,000 people and has both familial and sporadic etiologies. Only about ten percent of affected individuals have the familial form; however, familial and sporadic ALS cases share common pathological features (Haverkamp et al., Brain 118:707-719, 1995; Hayashi et al., J. Neurol. Sci. 105:73-78, 1991), suggesting that both genetic and environmental factors contribute to disease development.

Currently, the best animal model available for ALS is based on mutation of the superoxide dismutase-1 (SOD1) gene (Rosen et al., Nature 362:59-62, 1993; see erratum at Rosen et al., Nature 364:36241, 1993). Introduction of various human SOD1 mutant genes into transgenic mice leads to degeneration of motor neurons (Bruijn et al., Neuron 18:327-338, 1997; Del Canto and Gurney, Am. J. Pathol. 145:1271-1279, 1994; Gurney et al., Science 264:1772-1775, 1994; Ripps et al., Proc. Natl. Acad. Sci. USA 92:689-693, 1995; Wong et al., Neuron 14:1105-1116, 1995), and expression of SOD1 mutant protein in both glial cells and motor neurons contributes differently to disease pathogenesis (Boillee et al., Science 312:1389-1392, 2006; Clement et al., Science 302:113-117, 2003). Disease onset in these mice varies from three to eight months of age, possibly influenced more by transgene copy number (Alexander et al., Brain Res. Mol. Brain. Res. 130:7-15, 2004) than by mutation site, and evolves rapidly. The pattern of motor neuron degeneration observed shares many features with ALS in humans, including axonal swelling, vacuolation associated with swelling mitochondria in cell bodies and dendrites, neurofibrils associated with phosphorylated neurofilament (NF), and numerous cellular inclusions positive for hyaline and ubiquitin. Interestingly, transgenic mice expressing wild-type human SOD1 (Wong et al., Neuron 14:1105-1116, 1995) or SOD1 null mice (Reaume et al., Nature Genet. 13:43-47, 1996) do not exhibit motor neuron degeneration, suggesting the mutant gene is a gain-of-function mutation.

Disease progression of SOD1 mutant mice has been studied in detail, particularly SOD1 (G93A) mutant mice (Gurney et al., Science 264:1772-1775, 1994; Wang and Zhang, Eur. J. Neurosci. 22:2376-2380, 2005). SOD1 (G93A) mutant mice express a high copy number of the mutant gene and exhibit earlier onset (about 90 days) and shorter lifespan (about 140 days) than other mutant lines. The earliest pathological changes seen in these mice are mitochondrial swelling, first detectable around postnatal day 30 (P30) by electron microscopy and then around day P50-60 by light microscopy (Bendotti et al., J. Neurol. Sci. 191:25-33, 2001; Dal Canto and Gurney, Am. J. Pathol. 145:1271-1279, 1994; Kong and Xu, J. Neurosci. 18:3241-3250, 1998; Martin et al., J. Comp. Neurol. 500:20-46, 2006). Another detectable early change is degeneration of motor neuron axons. The largest axons innervating fast muscle fibers are the earliest to degenerate at around P50 (Frey et al., J. Neurosci. 20:2534-2542, 2000; Pun et al., Nat. Neurosci. 9:408-419, 2006). Consistent with axonal degeneration, abnormal muscle activities are also detected by electromyogram (EMG) as early as P60 (Kennel et al., Neuroreport 7:1427-1431, 1996; Miana-Mena et al., Amyotroph Lateral Scler. Other Motor Neuron Disord. 6:55-62, 2005). Besides these morphological and physiological changes, expression of several molecules also increases in pre-symptomatic stages, including p38 MAP kinase, nNOS, AKT MAP kinase, caspase-3, and cytokines like TNF-α, TGF-β, and m-CSF (Elliot, Brain Res. Mol. Brain. Res. 95:172-178, 2001; Wengenack et al., Brain Res. 1027:73-86, 2004). On the other hand, activation of astrocytes and microglia occurs at disease onset and becomes more prevalent at later stages (Hall et al., Glia 23:249-256, 1998; Wengenack et al., Brain Res. 1027:73-86, 2004). These data suggest that the disease originates from pathological changes in motor neurons occurring long before clinical symptoms are evident and affect neighboring glial cells. Therefore, understanding pre-symptomatic events is critical for treatment.

Currently, effective biomarkers, especially general biomarkers, are lacking for ALS and other neurodegenerative diseases (Rachakonda et al., Cell Res. 14:347-358, 2004). Many attempts to identify disease-specific biomarkers have focused on genetic mutations in APP or presenilin for Alzheimer's Disease (AD) or α-synuclein or Parkin for Parkinson's disease (PD). While these biomarkers are definitive and accurate, they apply to only a small percentage of inherited cases, as most degenerative cases (80-95 percent) are sporadic. Besides, as these biomarkers are expressed before birth and do not necessarily indicate the health of the brain, their use in humans may face ethical considerations such as screening tools in selection of birth, marriage and employment. Thus, markers such as Aβ42 and total tau protein for AD and α-synuclein protein for PD have been analyzed from blood and cerebrospinal fluids (CSF). However, these biomarkers are usually breakdown products of abnormal proteins expressed in late disease stages and do not indicate early damage. They also provide little information as to the injury site. Therefore, the most promising biomarkers are those detected by in vivo imaging, such as PET imaging of dopamine transport for PD or neurofibrillary tangles and senile amyloid plaques for AD. While the imaging resolution for these biomarkers continues to improve, these markers are apparent only at late disease states. Therefore, further research is necessary until markers useful for early detection with non-invasive methods are identified.

Also lacking are incentives to investigate general biomarkers that are common to neural cell injury seen in several neurodegenerative diseases. Most studies of available biomarkers focus on inherited causal factors of a specific disease, which may not apply to other diseases. Also, most biomarkers identified thus far do not show obvious links to the health of neural cells in affected brain areas and are not very useful to monitor disease progression or assess recovery during treatment. Therefore, different strategies and emphases are needed to identify general biomarkers, allowing development of more efficient and more broadly applicable methods for diagnosing and monitoring treatments of neurodegenerative disease.

Several general biomarkers have been investigated based on common cellular stress responses occurring during these diseases. For example, some markers of oxidative stress such as malondialdehyde, superoxide radicals, and 8-hydroxyguanosine are detected in blood of PD patients (Michell et al., Brain 127:1693-1705, 2004) Elevated homocysteine levels are also seen in both AD and PD patients (O'Suilleabhain et al., Arch. Neurol. 61:865-868, 2004). Some pro-inflammatory factors like tumor necrosis factor (TNF-α) are also found in PD (Le et al., Arch. Neurol. 56:194-200, 1999). High levels of glutamate, a risk factor for excitotoxicity, are linked to ALS (Rothstein, Neurology 47:S19-25, 1996, discussion S26; Shaw and Ince, J. Neurology 244:S3-S14, 1997). Nevertheless, none of these general markers show sufficient sensitivity and spatial resolution to be effective in disease detection in animals or humans. Also, most are bioproducts produced at late disease stages and are not suitable for early detection.

Thus, there is a need for biomarkers for neurodegenerative diseases such as ALS.

The present invention meets this and other needs.

### SUMMARY OF THE INVENTION

The present invention provides biomarkers for detecting neurodegeneration in an individual. Such biomarkers that have a two-fold or greater difference in expression in the spinal cord of 60- and/or 100-day-old SOD1 mutant mice compared with the expression of the biomarkers in wild-type littermates and thus detect early stages of neurodegeneration resulting from injury or disease.

According to one aspect of the invention, methods are provided for detection of neurodegeneration in an individual (including humans and non-human animals) comprising providing a sample from the individual comprising a neural cell (e.g., a neuron or glial cell); detecting levels of a biomarker polypeptide or polynucleotide in the sample comprising the polypeptide; and comparing the levels of the biomarker polypeptide or polynucleotide in the sample to levels of the biomarker polypeptide in a control sample; wherein expression of the biomarker polypeptide or polynucleotide has a two-fold or greater difference in expression in the spinal cord of 60- and/or 100-day-old SOD1 mutant mice compared with expression wild-type littermates. Biomarkers useful for such methods include but are not limited to the following: Usp18, Ifi202, Iigp1, Ifi27, Ifit3, Oas12, Ifit1, Rsad2, Ifi44, Isg15, Cxcl10, Gbp2, Socs3, Irf8, Oas1a, Irgm, B2m, Psmb8, Igtp, Isgf3g, Ifitm3, Stat1, Ifih1, Iigp2, Ifit2, Samhd1, and Clec7a polynucleotides and their corresponding polypeptides. Such methods are useful for detecting neurodegeneration that results, for example, from such diseases and injuries as Alzheimer's disease, Parkinson's disease, amyotrophic lateral sclerosis, Huntington's disease, encephalitis and dementia resulting from infection with Human Immunodeficiency Virus, brain ischemia, stroke, trauma, viral infection and prion infection

According to one embodiment, such methods of comprise contacting the sample with an antibody (including but not limited to a monoclonal antibody) that binds selectively to a biomarker polypeptide, and detecting binding of the antibody to the biomarker polypeptide, as, for example, in an ELISA assay or a bio-barcode assay.

According to another embodiment, such methods comprise contacting the sample with a first primer that comprises the polynucleotide sequence that hybridizes selectively to the biomarker polynucleotide and a second primer comprising a polynucleotide sequence that hybridizes to the biomarker polynucleotide, performing an amplification reaction (for example, performing a polymerase chain reaction [PCR], including but not limited to quantitative PCR), and quantitating an amplification product of the biomarker polynucleotide in the sample. An alternative embodiment involves performing a bio-barcode assay for the polynucleotide.

According to another embodiment, methods are provided for assessing the effectiveness of a course of treatment for an individual suffering from a neurodegenerative disease or neural cell damage, the method comprising (a) measuring a first level of a biomarker polypeptide or polynucleotide in a sample from the individual at a first time point during the course of treatment, (b) measuring a second level of the biomarker polypeptide or polynucleotide in a sample from the individual at a second time point during the course of treatment, and (c) comparing the measurements of the biomarker polypeptide or polynucleotide at the first timepoint and the second timepoint; wherein expression of the biomarker polypeptide or polynucleotide has a two-fold or greater difference in expression in the spinal cord of 60- and/or 100-day-old SOD1 mutant mice compared with expression wild-type littermates. Similarly, according to another embodiment, methods are provided for assessing the progression of a neurodegenerative disease in an individual suffering from the neurodegenerative disease or neural cell damage, the method comprising (a) measuring a first level of a biomarker polypeptide or polynucleotide in a sample from the individual at a first time point during the course of treatment, (b) measuring a second level of the biomarker polypeptide or polynucleotide in a sample from the individual at a second time point during the course of treatment, and (c) comparing the measurements of the biomarker polypeptide or polynucleotide at the first timepoint and the second timepoint; wherein expression of the biomarker polypeptide or polynucleotide has a two-fold or greater difference in expression in the spinal cord of 60- and/or 100-day-old SOD1 mutant mice compared with expression wild-type littermates.

According to another aspect of the invention, methods are provided for identifying a molecule for detecting neurodegeneration or neural cell damage in an individual, such methods comprising: (1) providing a sample from the individual comprising a biomarker polypeptide; (2) contacting the sample with a test molecule; (3) determining whether the test molecule binds to, or is bound by, the biomarker polypeptide; wherein expression of the biomarker polynucleotide has a two-fold or greater difference in expression in the spinal cord of 60- and/or 100-day-old SOD1 mutant mice compared with expression wild-type littermates. Such methods may optionally also comprise determining whether the test molecule crosses the blood-brain barrier.

Any of the foregoing methods may be automated.

### BRIEF DESCRIPTION OF THE FIGURES

FIG. 1 shows the DNA sequence for the Mus musculus Isg15 ubiquitin-like modifier (Isg15) mRNA (accession number NM_{—}015783 in the NCBI RefSeq database, http://www.ncbi.nlm.nih.gov).

FIG. 2 shows the amino acid sequence for Isg15.

FIG. 3 shows the results of quantitative RT-PCR (Q RT-PCR) analysis of eight candidate ISGs. The ratio of SOD1/WT was used to indicate the expression level of each gene. A. the Q RT-PCR data showed a good concordance with the microarray results. All eight candidate genes had significantly higher levels of mRNA in the total spinal cord from SOD1 mice than in that from wild-type littermate. B. Seven of eight genes showed significantly higher levels of their mRNAs in the ventral grey matter than in the whole spinal cord of SOD1 mice. C. the mRNA levels for individual genes varied extensively in the ventral grey matter at different disease stages.

### DETAILED DESCRIPTION OF THE INVENTION

### Definitions and Methods

The following definitions and methods are provided to better define the present invention and to guide those of ordinary skill in the art in the practice of the present invention. Unless otherwise noted, terms are to be understood according to conventional usage by those of ordinary skill in the relevant art. Definitions of common terms in molecular biology may also be found in Rieger et al., Glossary of Genetics: Classical and Molecular, 5th edition, Springer-Verlag: New York, 1991; and Lewin, Genes V, Oxford University Press: New York, 1994. The nomenclature for DNA bases as set forth at 37 CFR 1.822 is used. The standard one- and three-letter nomenclature for amino acid residues is used.

### Polynucleotides

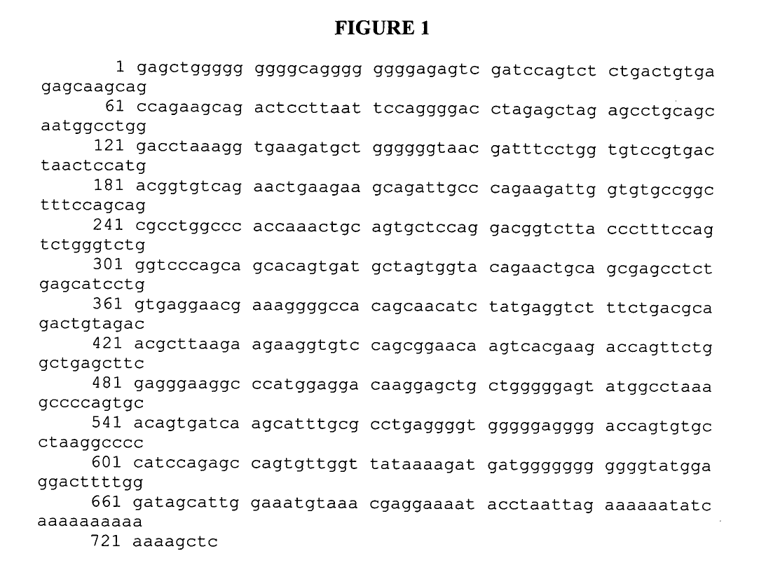

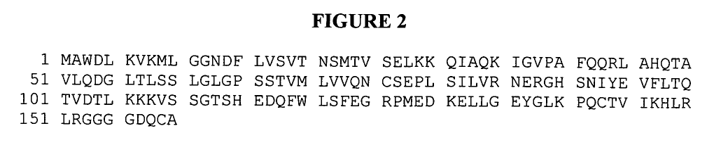

As used herein, the term “biomarker polynucleotide” refers to a polynucleotide (or probe or primer) that comprises a polynucleotide sequence from a gene that has a two-fold or greater difference in gene expression in the spinal cord of 60-, or 100-, or both 60- and 100-day-old SOD1 (G93A) mutant mice compared with wild-type littermates, as discussed in the Examples. We identified 45 biomarker polynucleotides that have a two-fold or greater difference in gene expression in the spinal cord of 60- and/or 100-day-old SOD1 mutant mice. Representative biomarker polynucleotides are included in Table 1 together with their respective accession numbers in the National Center for Biotechnology Information (NCBI) Reference Sequence (RefSeq) database (http://www.ncbi.nlm.nih.gov), which provides nucleotide and/or amino acid sequence information for each biomarker polynucleotide. The nucleotide sequences for one of these representative biomarker polynucleotides, Isg15, as well as the sequence of the Isg15 polypeptide, are shown in FIGS. 1 and 2, respectively. Sequences for each of the biomarker polynucleotides listed in Table 1 can be found at the RefSeq database using the corresponding accession number in Table 1.

Table 1: Representative Biomarker Genes and their Accession Numbers in the NCBI RefSeq Database

TABLE 1
Representative biomarker genes and their accession numbers
in the NCBI RefSeq database

| Genes | RefSeq Accession # |
|---|---|
| Usp18: ubiquitin specific peptidase 18 | NM_011909 |
| | NM_008327 |
| Ifi202: interferon activated gene 202 | NM_011940 |
| Iigp1: interferon inducible GTPase 1 | NM_021792 |
| Ifi27: interferon, alpha-inducible | NM_029803 |
| protein 27 | |
| Ifit3: interferon-induced protein with | NM_010501 |
| tetratricopeptide repeats 3 | |
| Oasl2: 2′-5′ oligoadenylate synthetase- | NM_011854 |
| like 2 | |
| Ifit1: interferon-induced protein with | NM_008331 |
| tetratricopeptide repeats 1 | |
| Rsad2: radical S-adenosyl methionine | NM_021384 |
| domain containing 2 | |
| Ifi44: interferon-induced protein 44 | NM_133871 |
| Isg15: interferon, alpha-inducible | NM_015783 |
| protein | |
| Cxcl10: chemokine (C—X—C motif) | NM_021274 |
| ligand 10 | |
| Gbp2: guanylate nucleotide binding | NM_010260 |
| protein 2 | |
| Socs3: suppressor of cytokine | NM_007707 |
| signaling 3 | |
| Irf8: interferon regulatory factor 8 | NM_008320 |
| Oas1a: 2′-5′ oligoadenylate synthetase | NM_145211 |
| 1A | |
| Irgm: immunity-related GTPase | NM_008326 |
| family, M | |
| B2m: beta-2 microglobulin | NM_009735 |
| Psmb8: proteosome (prosome, | NM_010724 |
| macropain) subunit, beta type 8 (large | |
| multifunctional peptidase 7) | |
| Igtp: interferon gamma induced | NM_018738 |
| GTPase | |
| Isgf3g: interferon dependent positive | NM_008394 |
| acting transcription factor 3 gamma | |
| Ifitm3: interferon induced | NM_025378 |
| transmembrane protein 3 | |
| Stat1: signal transducer and activator | NM_009283 |
| of transcription 1 | |
| Ifih1: interferon induced with helicase | NM_027835 |
| C domain 1 | |
| Iigp2: interferon inducible GTPase 2 | NM_019440 |
| Ifit2: interferon-induced protein with | NM_008332 |
| tetratricopeptide repeats 2 | |
| Samhd1: SAM domain and HD | NM_018851 |
| domain, 1 | |
| Clec7a: C-type lectin domain family 7, | NM_020008 |
| member a | |

As used herein, the term “biomarker polynucleotide” refers to genomic DNA, mRNA, and cDNA corresponding to each biomarker polynucleotide, including but not limited to the protein-coding region thereof. Also encompassed by the term “biomarker polynucleotides” are, for example: fragments or portions of an mRNA or cDNA for a particular biomarker gene, including but not limited to fragments that encode antigenic determinants of a polypeptide encoded by such polynucleotides (e.g., those that elicit antibodies that bind selectively to such polypeptides); probes and primers that hybridize selectively to biomarker polynucleotides; etc. Also included are mutated or variant polynucleotides that include one or more nucleotide insertions, deletions, or substitutions from the wild-type biomarker polynucleotide sequence, but that, for example: retain the ability to bind selectively to a biomarker polynucleotide; encode a polypeptide that includes an antigenic determinant of a polypeptide encoded by a biomarker polynucleotide; encode a polypeptide having a biological activity of a polypeptide encoded by a biomarker polynucleotide; etc.

As used herein, the term “hybridizes selectively” refers to binding of a probe, primer or other polynucleotide, under stringent hybridization conditions, to a target polynucleotide, such as a native, or wild-type, biomarker mRNA or cDNA, to a substantially higher degree than to other polynucleotides. In particular, a probe that “hybridizes selectively” to a biomarker polynucleotide does not hybridize substantially to a polynucleotide other than a biomarker polynucleotide under stringent hybridization conditions and therefore can be used to distinguish a biomarker polynucleotide from other polynucleotides. Similarly, a primer that “hybridizes selectively” to a biomarker polynucleotide, when used in an amplification reaction such as PCR, results in amplification of a target biomarker polynucleotide without resulting in substantial amplification of other polynucleotides under suitable amplification conditions. Thus, all or substantially all of a biomarker polynucleotide-selective probe or primer hybridizes to the target biomarker polynucleotide under suitable conditions, as can be determined given the sensitivity of a particular procedure. Similarly, as used herein, the term “selective for” in reference to a polynucleotide, indicates that the polynucleotide hybridizes selectively to a target polynucleotide.

As used herein, the terms “wild-type” or “native” in reference to a polynucleotide are used interchangeably to refer to a polynucleotide that has 100% sequence identity with a reference polynucleotide that can be found in a cell or organism, or a fragment thereof.

As noted above, the nucleotide sequence for each of the biomarker polynucleotides of the invention is provided at the NCBI RefSeq database. Polynucleotide (e.g., DNA or RNA) sequences for each biomarker polynucleotide also may be determined by sequencing a polynucleotide molecule using an automated DNA sequencer. A polynucleotide sequence determined by this automated approach can contain some errors. The actual sequence can be confirmed by resequencing the polynucleotide by automated means or by manual sequencing methods well known in the art.

Unless otherwise indicated, each “nucleotide sequence” set forth herein is presented as a sequence of deoxyribonucleotides (abbreviated A, G, C and T). However, the term “nucleotide sequence” of a DNA molecule as used herein refers to a sequence of deoxyribonucleotides, and for an RNA molecule, the corresponding sequence of ribonucleotides (A, G, C and U) where each thymidine deoxynucleotide (T) in the specified deoxynucleotide sequence in is replaced by the ribonucleotide uridine (U).

By “isolated” polynucleotide is intended a polynucleotide that has been removed from its native environment For example, recombinant polynucleotides contained in a vector are considered isolated for the purposes of the present invention. Further examples of isolated polynucleotides include recombinant polynucleotides maintained in heterologous host cells or purified (partially or substantially) polynucleotides in solution. Isolated RNA molecules include in vivo or in vitro RNA transcripts of the DNA molecules of the present invention. Isolated polynucleotides according to the present invention further include such molecules produced synthetically.

Polynucleotides can be in the form of RNA, such as mRNA, or in the form of DNA, including, for instance, cDNA and genomic DNA. The DNA can be double-stranded or single-stranded. A single-stranded DNA or RNA can be a coding strand, also known as the sense strand, or it can be a non-coding strand, also referred to as the anti-sense strand. Polynucleotides can include non-naturally occurring nucleotide or ribonucleotide analogs.

The term “fragment” (of a polynucleotide) as used herein refers to polynucleotides that are part of a longer polynucleotide having a length of at least about 15, 20, 25, 30, 35, or 40 nucleotides (nt), and which are useful, for example, as probes and primers. Thus, for example, a fragment of a biomarker polynucleotide at least 20 nucleotides in length includes 20 or more contiguous bases from the nucleotide sequence of the biomarker polynucleotide. Such DNA fragments may be generated by the use of automated DNA synthesizers or by restriction endonuclease cleavage or shearing (e.g., by sonication) a full-length biomarker polynucleotide, for example.

Also encompassed by the present invention are isolated polynucleotides that hybridize under stringent hybridization conditions to a biomarker polynucleotide such as, for example, an mRNA. By “stringent hybridization conditions” is intended overnight incubation at 42° C. in a solution comprising: 50% formamide, 5×SSC (750 mM NaCl, 75 mM trisodium citrate), 50 mM sodium phosphate (pH7.6), 5×Denhardt's solution, 10% dextran sulfate, and 20 μg/ml denatured, sheared salmon sperm DNA, followed by washing the filters in 0.1×SSC at about 65° C. Alternatively, stringent hybridizations are conditions used for performance of a polymerase chain reaction (PCR). Such hybridizing polynucleotides are useful diagnostically as a probe according to conventional DNA hybridization techniques or as primers for amplification of a target sequence by the polymerase chain reaction (PCR).

As used herein, the term “hybridizes (or binds) specifically” is used interchangeably with the term “hybridizes (or binds) selectively” means that most or substantially all hybridization of a probe or primer is to a particular polynucleotide in a sample under stringent hybridization conditions.

The present invention also provides polynucleotides that encode all or a portion of a polypeptide, e.g., a full-length biomarker polypeptide or a portion thereof. Such protein-coding polynucleotides may include, but are not limited to, those sequences that encode the amino acid sequence of the particular polypeptide or fragment thereof and may also include together with additional, non-coding sequences, including for example, but not limited to introns and non-coding 5′ and 3′ sequences, such as the transcribed, non-translated sequences that play a role in transcription, mRNA processing—including splicing and polyadenylation signals, e.g., ribosome binding and stability of mRNA; an additional coding sequence which codes for additional amino acids, such as those which provide additional functionalities. In addition, the sequence encoding the polypeptide can be fused to a heterogeneous polypeptide or peptide sequence, such as, for example a marker sequence that facilitates purification of the fused polypeptide. One example of such a marker sequence is a hexa-histidine peptide, such as the tag provided in a pQE vector (Qiagen, Inc.). As described in Gentz et al., Proc. Natl. Acad. Sci. USA 86:821-824 (1989), for instance, hexa-histidine provides for convenient purification of the fusion protein. The “HA” tag is another peptide useful for purification which corresponds to an epitope derived from the influenza hemagglutinin (HA) protein, which has been described by Wilson et al., Cell 37:767 (1984).

The present invention further relates to variants of the native, or wild-type, biomarker polynucleotides of the present invention, which encode portions, analogs or derivatives of a biomarker polypeptide (as defined below). Variants can occur naturally, such as a natural allelic variant, i.e., one of several alternate forms of a gene occupying a given locus on a chromosome of an organism. Non-naturally occurring variants can be produced, e.g., using known mutagenesis techniques or by DNA synthesis. Such variants include those produced by nucleotide substitutions, deletions or additions. The substitutions, deletions or additions can involve one or more nucleotides. The variants can be altered in coding or non-coding regions or both. Alterations in the coding regions can produce conservative or non-conservative amino acid substitutions, deletions or additions. Also included are silent substitutions, additions and deletions, which do not alter the properties and activities of the biomarker polypeptide or portions thereof.

Further embodiments of the invention include isolated polynucleotide molecules have, or comprise a sequence having, a high degree of sequence identity with a native, or wild type, biomarker polynucleotide, for example, at least 90%, 95%, 96%, 97%, 98% or 99% identical thereto.

A polynucleotide is considered to have a nucleotide sequence at least, for example, 95% “identical” to a reference nucleotide sequence if it is identical to the reference sequence except that it includes up to five mutations (additions, deletions, or substitutions) per each 100 nucleotides of the reference nucleotide sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence. Nucleotide sequence identity may be determined conventionally using known computer programs such as the BESTFIT program (Wisconsin Sequence Analysis Package, Version 8 for Unix, Genetics Computer Group, University Research Park, 575 Science Drive, Madison, Wis. 53711. BESTFIT uses the local homology algorithm of Smith and Waterman, Adv. Appl. Math. 2:482-489 (1981), to find the best segment of homology between two sequences. When using BESTFIT or any other sequence alignment program to determine whether a particular sequence is, for instance, 95% identical to a reference sequence according to the present invention, the parameters are set, of course, such that the percentage of identity is calculated over the full length of the reference nucleotide sequence and that gaps in homology of up to 5% of the total number of nucleotides in the reference sequence are allowed.

### Recombinant Constructs: Vectors and Host Cells

The present invention also provides recombinant polynucleotide constructs that comprise a biomarker polynucleotide, including but not limited to vectors. The present invention also provides host cells comprising such vectors and the production of biomarker polypeptides or fragments thereof by recombinant or synthetic techniques.

“Operably Linked”. A first nucleic-acid sequence is “operably linked” with a second nucleic-acid sequence when the first nucleic-acid sequence is placed in a functional relationship with the second nucleic-acid sequence. For instance, a promoter is operably linked to a coding sequence if the promoter affects the transcription or expression of the coding sequence. Generally, operably linked DNA sequences are contiguous and, where necessary to join two protein coding regions, in reading frame.

“Recombinant”. A “recombinant” polynucleotide is made by an artificial combination of two otherwise separated segments of sequence, e.g., by chemical synthesis or by the manipulation of isolated segments of polynucleotides by genetic engineering techniques. Techniques for nucleic-acid manipulation are well-known (see, e.g., Sambrook et al., 1989, and Ausubel et al., 1992). Methods for chemical synthesis of polynucleotides are discussed, for example, in Beaucage and Carruthers, Tetra. Letts. 22:1859-1862, 1981, and Matteucci et al., J. Am. Chem. Soc. 103:3185, 1981. Chemical synthesis of polynucleotides can be performed, for example, on commercial automated oligonucleotide synthesizers.

Recombinant vectors are produced by standard recombinant techniques and may be introduced into host cells using well known techniques such as infection, transduction, transfection, transvection, electroporation and transformation. The vector may be, for example, a phage, plasmid, viral or retroviral vector. Retroviral vectors may be replication competent or replication defective. In the latter case, viral propagation generally will occur only in complementing host cells.

Expression vectors include sequences that permit expression of a polypeptide encoded by a polynucleotide of interest in a suitable host cell. Such expression may be constitutive or non-constitutive, e.g., inducible by an environmental factor or a chemical inducer that is specific to a particular cell or tissue type, for example. Expression vectors include chromosomal-, episomal- and virus-derived vectors, e.g., vectors derived from bacterial plasmids, bacteriophage, yeast episomes, yeast chromosomal elements, viruses such as baculoviruses, papova viruses, vaccinia viruses, adenoviruses, fowl pox viruses, pseudorabies viruses and retroviruses, and vectors derived from combinations thereof, such as cosmids and phagemids.

In expression vectors, a polynucleotide insert is operably linked to an appropriate promoter. The promoter may be a homologous promoter, i.e., a promoter or functional portion thereof, that is associated with the polynucleotide insert in nature. Alternatively, the promoter may be a heterologous promoter, i.e., a promoter or functional portion thereof, that is not associated with the polynucleotide insert in nature, for example, a bacterial promoter used for high-level protein expression in bacterial cells operably linked to a protein-coding region. The expression constructs will further contain sites for transcription initiation, termination and, in the transcribed region, a ribosome binding site for translation. The coding portion of the mature transcripts expressed by the constructs will include a translation initiating AUG at the beginning and a termination codon appropriately positioned at the end of the polypeptide to be translated.

Vectors may include one or more selectable marker suitable for selection of a host cell into which such a vector has been introduced. Such markers include dihydrofolate reductase or neomycin resistance for eukaryotic cell culture and tetracycline or ampicillin resistance genes for culturing in E. coli and other bacteria. Representative examples of appropriate hosts include bacterial cells, such as E. coli, Streptomyces and Salmonella typhimurium cells; fungal cells, such as yeast cells; insect cells such as Drosophila S2 and Spodoptera Sf9 cells; animal cells such as CHO, COS and Bowes melanoma cells; and plant cells. Appropriate culture media and conditions for the above-described host cells are known in the art.

Bacterial promoters suitable include the E. coli lacI and lacZ promoters, the T3 and T7 promoters, the gpt promoter, the lambda PR and PL promoters and the trp promoter. Eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

A polypeptide of interest may be expressed in a modified form, such as a fusion protein, and may include not only secretion signals but also additional heterologous functional regions. For instance, a region of additional amino acids, particularly charged amino acids, may be added to the N-terminus of the polypeptide to improve stability and persistence in the host cell, during purification or during subsequent handling and storage. Also, peptide moieties may be added to the polypeptide to facilitate purification. Such regions may be removed prior to final preparation of the polypeptide. The addition of peptide moieties to polypeptides to engender secretion or excretion, to improve stability and to facilitate purification, among others, are familiar and routine techniques in the art.

An expressed polypeptide of interest can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography.

Polypeptides of the present invention include naturally purified products, products of chemical synthetic procedures, and products produced by recombinant techniques from a prokaryotic or eukaryotic host, including, for example, bacterial, yeast, higher plant, insect and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes.

### Polypeptides

As used herein, the phrase “an biomarker polypeptide” refers to a polypeptide at least 10, 11, 12, 12, 14, 15, 20, 30, 40, 49, 50, 100 or more amino acid residues in length and having a high degree of sequence identity with a full-length native, or wild-type, polypeptide encoded by a biomarker polynucleotide (or the full-length version of each biomarker polynucleotide as it exists in the genome or a human or other animal of interest). Included are variant forms of biomarker polypeptides that include deletions, insertions or substitutions of one or more amino acid residues in a native biomarker polypeptide sequence, including without limitation polypeptides that exhibit activity similar, but not necessarily identical, to an activity of the full-length native, or wild-type, biomarker polypeptide or fragment thereof as measured in a relevant biological assay.

As used herein, the terms “wild-type” or “native” in reference to a peptide or polypeptide are used interchangeably to refer to a polypeptide that has 100% sequence identity with a reference polypeptide that can be found in a cell or organism, or a fragment thereof.

As used herein, the terms “peptide” and “oligopeptide” are considered synonymous and, as used herein, each term refers to a chain of at least two amino acids coupled by peptidyl linkages. As used herein, the terms “polypeptide” and “protein” are considered synonymous and each term refers to a chain of more than about ten amino acid residues. All oligopeptide and polypeptide formulas or sequences herein are written from left to right and in the direction from amino terminus to carboxy terminus.

As used herein, the term “isolated” polypeptide or protein refers to a polypeptide or protein removed from its native environment. For example, recombinantly produced polypeptides and proteins expressed in host cells are considered isolated for purposes of the invention as are native or recombinant polypeptides and proteins which have been substantially purified by any suitable technique.

As used herein, the term “binds selectively” is interchangeable with the term “binds specifically, and, when used in reference to a biomarker polypeptide, refers to binding of an antibody, ligand, receptor, substrate, or other binding agent to the target biomarker polypeptide to a substantially higher degree than to other polypeptides. According to some embodiments, all or substantially all binding of an antibody or other binding agent is to the target biomarker polypeptide, as can be determined given the sensitivity of a particular procedure. An antibody, ligand, receptor, substrate or other binding agent is said to be “selective for” or specific for” a polypeptide or other target molecule if it binds selectively to the target molecule.

The amino acid sequence of a biomarker polypeptide or peptide can be varied without significant effect on the structure or function of the protein. In general, it is possible to replace residues which contribute to the tertiary structure of the polypeptide or peptide, provided that residues performing a similar function are used. In other instances, the type of residue may be completely unimportant if the alteration occurs at a non-critical region of the protein.

Thus, the invention further includes variations of a biomarker polypeptide or peptide that show substantial activity of the wild-type or native biomarker polypeptide. Such mutants include deletions, insertions, inversions, repeats, and type substitutions (for example, substituting one hydrophilic residue for another, but not strongly hydrophilic for strongly hydrophobic as a rule). Small changes or such “neutral” amino acid substitutions will generally have little effect on activity.

Typically seen as conservative substitutions are the replacements, one for another, among the aliphatic amino acids Ala, Val, Leu and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr.

Guidance concerning which amino acid changes are likely to be phenotypically silent (i.e., are not likely to have a significant deleterious effect on a function) can be found, for example, in Bowie et al., Science 247:1306-1310, 1990.

Thus, a fragment, derivative or analog of a native, or wild-type biomarker polypeptide, may be (i) one in which one or more of the amino acid residues are substituted with a conserved or non-conserved amino acid residue and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide, such as an IgG Fc fusion region peptide or leader or secretory sequence or a sequence that is employed for purification of the mature polypeptide or a proprotein sequence.

Charged amino acids may be substituted with another charged amino acid. Charged amino acids may also be substituted with neutral or negatively charged amino acids, resulting in proteins with reduced positive charge. The prevention of aggregation is highly desirable to avoid a loss of activity and increased immunogenicity (Pinckard et al., Clin Exp. Immunol. 2:331-340, 1967; Robbins et al., Diabetes 36:838-845, 1987; Cleland et al., Crit. Rev. Therapeutic Drug Carrier Systems 10:307-377, 1993).

The replacement of amino acids can also change the selectivity of protein binding to cell surface receptors. Ostade et al., Nature 361:266-268 (1993) describes certain mutations resulting in selective binding of TNF-α to only one of the two known types of TNF receptors.

It is well known in the art that one or more amino acids in a native sequence can be substituted with other amino acid(s), the charge and polarity of which are similar to that of the native amino acid, i.e., a conservative amino acid substitution, resulting in a silent change. Conservative substitutes for an amino acid within the native polypeptide sequence can be selected from other members of the class to which the amino acid belongs. Amino acids can be divided into the following four groups: (1) acidic amino acids, (2) basic amino acids, (3) neutral polar amino acids, and (4) neutral, nonpolar amino acids. Representative amino acids within these various groups include, but are not limited to, (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, cystine, tyrosine, asparagine, and glutamine; and (4) neutral nonpolar (hydrophobic) amino acids such as alanine, leucine, isoleucine, valine, proline, phenylalanine, tryptophan, and methionine. Conservative amino acid substitution within the native polypeptide sequence can be made by replacing one amino acid from within one of these groups with another amino acid from within the same group. In one aspect, biologically functional equivalents of the proteins or fragments thereof of the present invention can have ten or fewer, seven or fewer, five or fewer, four or fewer, three or fewer, two, or one conservative amino acid changes. The encoding nucleotide sequence will thus have corresponding base substitutions, permitting it to encode biologically functional equivalent forms of the proteins or fragments of the present invention.

It is understood that certain amino acids may be substituted for other amino acids in a protein structure without appreciable loss of interactive binding capacity with structures such as, for example, antigen-binding regions of antibodies or binding sites on substrate molecules. Because it is the interactive capacity and nature of a protein that defines that protein's biological functional activity, certain amino acid sequence substitutions can be made in a protein sequence and, of course, its underlying DNA coding sequence and, nevertheless, a protein with like properties can still be obtained. It is thus contemplated by the inventors that various changes may be made in the peptide sequences of the proteins or fragments of the present invention, or corresponding DNA sequences that encode said peptides, without appreciable loss of their biological utility or activity. It is understood that codons capable of coding for such amino acid changes are known in the art.

In making such changes, the hydropathic index of amino acids may be considered. The importance of the hydropathic amino acid index in conferring interactive biological function on a protein is generally understood in the art (Kyte and Doolittle, J. Mol. Biol. 157:105-132, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of its hydrophobicity and charge characteristics (Kyte and Doolittle, J. Mol. Biol. 157:105-132, 1982); these are: isoleucine (+4.5), valine (+4.2), leucine (+3.8), phenylalanine (+2.8), cysteine/cystine (+2.5), methionine (+1.9), alanine (+1.8), glycine (−0.4), threonine (−0.7), serine (−0.8), tryptophan (−0.9), tyrosine (−1.3), proline (−1.6), histidine (−3.2), glutamate (−3.5), glutamine (−3.5), aspartate (−3.5), asparagine (−3.5), lysine (−3.9), and arginine (4.5). In making such changes, the substitution of amino acids whose hydropathic indices may be within ±2, or ±1, or within ±0.5.

It is also understood in the art that the substitution of like amino acids can be made effectively on the basis of hydrophilicity. U.S. Pat. No. 4,554,101 states that the greatest local average hydrophilicity of a protein, as govern by the hydrophilicity of its adjacent amino acids, correlates with a biological property of the protein.

As detailed in U.S. Pat. No. 4,554,101, the following hydrophilicity values have been assigned to amino acid residues: arginine (+3.0), lysine (+3.0), aspartate (+3.0.+−.1), glutamate (+3.0.+−0.1), serine (+0.3), asparagine (+0.2), glutamine (+0.2), glycine (0), threonine (−0.4), proline (−0.5.+−0.1), alanine (−0.5), histidine (−0.5), cysteine (−1.0), methionine (−1.3), valine (−1.5), leucine (−1.8), isoleucine (−1.8), tyrosine (−2.3), phenylalanine (−2.5), and tryptophan (−3.4). In making changes to a native polypeptide or peptide sequence, the substitution of amino acids whose hydrophilicity values may be within ±2, or within ±1, or within ±0.5.

Of course, the number of amino acid substitutions a skilled artisan would make depends on many factors, including those described above. Generally speaking, the number of substitutions for any given biomarker polypeptide will not be more than 50, 40, 30, 20, 10, 5, 3, or 2.

Amino acids in the biomarker polypeptides of the present invention that are essential for function can be identified by methods known in the art, such as site-directed mutagenesis or alanine-scanning mutagenesis (Cunningham and Wells, Science 244:1081-1085, 1989). The latter procedure introduces single alanine mutations at every residue in the molecule. The resulting mutant molecules are then tested for biological activity such as in vitro or in vivo ligand or receptor binding or other characteristic biological activities. Sites that are critical for ligand-receptor binding can also be determined by structural analysis such as crystallization, nuclear magnetic resonance or photoaffinity labeling (Smith et al., J. Mol. Biol. 224:899-904, 1992; de Vos et al. Science 255:306-312, 1992).

The polypeptides and peptides of the present invention include native, or wild-type polypeptides and peptides, and polypeptides or peptide variants that are at least 75%, 80%, 85%, 90%, 95%, 96%, 97%, 98% or 99% identical to (or have such a degree of identity with) the native biomarker polypeptide and fragments thereof.

By a polypeptide having an amino acid sequence at least, for example, 95% “identical” to a reference amino acid sequence is intended that the amino acid sequence of the polypeptide is identical to the reference sequence except that the polypeptide sequence may include up to five amino acid alterations per each 100 amino acids of the reference amino acid sequence of the reference polypeptide. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino- or carboxy-terminal positions of the reference amino acid sequence or anywhere between those terminal positions, interspersed either individually among residues in the reference sequence or in one or more contiguous groups within the reference sequence.

As a practical matter, whether any particular polypeptide has a particular degree of amino acid sequence identity when compared to a reference polypeptide can be determined conventionally using known computer programs such the Bestfit program (Wisconsin Sequence Analysis Package, Version 8 for Unix, Genetics Computer Group, University Research Park, 575 Science Drive, Madison, Wis. 53711. When using Bestfit or any other sequence alignment program to determine whether a particular sequence is, for instance, 95% identical to a reference sequence according to the present invention, the parameters are set, of course, such that the percentage of identity is calculated over the full length of the reference amino acid sequence and that gaps in homology of up to 5% of the total number of amino acid residues in the reference sequence are allowed.

In another embodiment of the present invention, there are provided fragments of the polypeptides described herein. Such fragments include but are not limited to fragments that comprise, e.g., 4, 5, 6, 7, 8, 9, 10 or more contiguous amino acid residues of a biomarker polypeptide that include one or more antigenic determinants of the biomarker polypeptide, for example, those that elicit antibodies that bind selectively to a particular biomarker polypeptide. Also included are fragments of a biomarker polypeptide that bind, for example a ligand, substrate, product, agonist or antagonist of a biomarker polypeptide, including but not limited to the active site. The polypeptide fragments of the present invention can be used for numerous purposes, for example, to elicit antibody production in a mammal, as molecular weight markers on SDS-PAGE gels, on molecular sieve gel filtration columns, or in efforts to discover small molecules that bind specifically to a particular biomarker polypeptide, for example.

Polypeptides of the present invention can be used to raise polyclonal and monoclonal antibodies, which are useful in diagnostic assays for detecting biomarker expression or for other purposes. Further, such polypeptides can be used in the yeast two-hybrid system to “capture” binding proteins (Fields and Song, Nature 340:245-246, 1989).

In another aspect, the invention provides a peptide or polypeptide comprising an epitope-bearing portion of a biomarker polypeptide of the invention. The epitope of this polypeptide portion is an immunogenic or antigenic epitope of a polypeptide of the invention. An “immunogenic epitope” is defined as a part of a protein that elicits an antibody response when the whole protein is the immunogen. These immunogenic epitopes are believed to be confined to a few loci on the molecule. On the other hand, a region of a protein molecule to which an antibody can bind is defined as an “antigenic epitope.” The number of immunogenic epitopes of a protein generally is less than the number of antigenic epitopes. See, for instance, Geysen et al., Proc. Natl. Acad. Sci. USA 81:3998-4002, 1984).

As to the selection of peptides or polypeptides bearing an antigenic epitope (i.e., that contain a region of a protein molecule to which an antibody can bind), it is well known in that art that relatively short synthetic peptides that mimic part of a protein sequence are routinely capable of eliciting an antiserum that reacts with the partially mimicked protein. See, for instance, Sutcliffe et al., Science 219:660-666, 1983). Peptides capable of eliciting protein-reactive sera are frequently represented in the primary sequence of a protein, can be characterized by a set of simple chemical rules, and are confined neither to immunodominant regions of intact proteins (i.e., immunogenic epitopes) nor to the amino or carboxyl terminals. Peptides that are extremely hydrophobic and those of six or fewer residues generally are ineffective at inducing antibodies that bind to the mimicked protein; longer, soluble peptides, especially those containing proline residues, usually are effective (Sutcliffe et al., supra, at 661).

Antigenic epitope-bearing peptides and polypeptides of the invention are therefore useful to raise antibodies, including monoclonal antibodies, which bind selectively to a polypeptide of the invention. Thus, a high proportion of hybridomas obtained by fusion of spleen cells from donors immunized with an antigen epitope-bearing peptide generally secrete antibody reactive with the native protein (Sutcliffe et al., supra, at 663). The antibodies raised by antigenic epitope-bearing peptides or polypeptides are useful to detect the mimicked protein, and antibodies to different peptides may be used for tracking the fate of various regions of a protein precursor which undergoes post-translational processing. The peptides and anti-peptide antibodies may be used in a variety of qualitative or quantitative assays for the mimicked protein, for instance in competition assays since it has been shown that even short peptides (e.g., about 9 amino acids) can bind and displace the larger peptides in immunoprecipitation assays. See, for example, Wilson et al., Cell 37:767-778, 1984). The anti-peptide antibodies of the invention also are useful for protein purification, e.g., by adsorption chromatography using known methods.

Antigenic epitope-bearing peptides and polypeptides of the invention designed according to the above guidelines may contain a sequence of at least 7, 8, 9, 10, 11, 12, 13, 14, 15, 20 or 30 or more amino acids contained within the amino acid sequence of a polypeptide of the invention. However, peptides or polypeptides comprising a larger portion of an amino acid sequence of a polypeptide of the invention, containing about 30 to about 50 amino acids, or any length up to and including the entire amino acid sequence of a polypeptide of the invention, also are considered epitope-bearing peptides or polypeptides of the invention and also are useful for inducing antibodies that react with the mimicked protein.

The amino acid sequence of the epitope-bearing peptide may be selected to provide substantial solubility in aqueous solvents (i.e., sequences including relatively hydrophilic residues and highly hydrophobic sequences may be avoided).

The epitope-bearing peptides and polypeptides of the invention may be produced by any conventional means for making peptides or polypeptides including recombinant means using nucleic acid molecules of the invention. For instance, a short epitope-bearing amino acid sequence may be fused to a larger polypeptide which acts as a carrier during recombinant production and purification, as well as during immunization to produce anti-peptide antibodies. Epitope-bearing peptides also may be synthesized using known methods of chemical synthesis. For instance, Houghten has described a simple method for synthesis of large numbers of peptides, such as 10-20 mg of 248 different 13 residue peptides representing single amino acid variants of a segment of the HA1 polypeptide which were prepared and characterized (by binding studies employing an enzyme-linked immunosorbent assay [ELISA]) in less than four weeks (Houghten, Proc. Natl. Acad. Sci. USA 82:5131-5135, 1985; and U.S. Pat. No. 4,631,211). In this procedure the individual resins for the solid-phase synthesis of various peptides are contained in separate solvent-permeable packets, enabling the optimal use of the many identical repetitive steps involved in solid-phase methods. A completely manual procedure allows 500-1000 or more syntheses to be conducted simultaneously.

Epitope-bearing peptides and polypeptides of the invention are used to induce antibodies according to methods well known in the art. See, for instance, Sutcliffe et al., supra; Wilson et al., supra; Chow et al., Proc. Natl. Acad. Sci. USA 82:910-914; and Bittle et al., J. Gen. Virol. 66:2347-2354, 1985). Generally, animals may be immunized with free peptide; however, anti-peptide antibody titer may be boosted by coupling of the peptide to a macromolecular carrier, such as keyhole limpet hemacyanin (KLH) or tetanus toxoid. For instance, peptides containing cysteine may be coupled to carrier using a linker such as m-maleimidobenzoyl-N-hydroxysuccinimide ester (MBS), while other peptides may be coupled to carrier using a more general linking agent such as glutaraldehyde. Animals such as rabbits, rats and mice are immunized with either free or carrier-coupled peptides, for instance, by intraperitoneal and/or intradermal injection of emulsions containing about 100 μg peptide or carrier protein and Freund's adjuvant. Several booster injections may be needed, for instance, at intervals of about two weeks, to provide a useful titer of anti-peptide antibody which can be detected, for example, by ELISA assay using free peptide adsorbed to a solid surface. The titer of anti-peptide antibodies in serum from an immunized animal may be increased by selection of anti-peptide antibodies, for instance, by adsorption to the peptide on a solid support and elution of the selected antibodies according to methods well known in the art.

Immunogenic epitope-bearing peptides of the invention, i.e., those parts of a protein that elicit an antibody response when the whole protein is the immunogen, are identified according to methods known in the art. For instance, Geysen et al. (1984), supra, discloses a procedure for rapid concurrent synthesis on solid supports of hundreds of peptides of sufficient purity to react in an enzyme-linked immunosorbent assay. Interaction of synthesized peptides with antibodies is then easily detected without removing them from the support. In this manner a peptide bearing an immunogenic epitope of a desired protein may be identified routinely by one of ordinary skill in the art. For instance, the immunologically important epitope in the coat protein of foot-and-mouth disease virus was located by Geysen et al. with a resolution of seven amino acids by synthesis of an overlapping set of all 208 possible hexapeptides covering the entire 213 amino acid sequence of the protein. Then, a complete replacement set of peptides in which all 20 amino acids were substituted in turn at every position within the epitope were synthesized, and the particular amino acids conferring specificity for the reaction with antibody were determined. Thus, peptide analogs of the epitope-bearing peptides of the invention can be made routinely by this method. U.S. Pat. No. 4,708,781 to Geysen (1987) further describes this method of identifying a peptide bearing an immunogenic epitope of a desired protein.

U.S. Pat. No. 5,194,392 to Geysen (1990) describes a general method of detecting or determining the sequence of monomers (amino acids or other compounds) which is a topological equivalent of the epitope (i.e., a “mimotope”) which is complementary to a particular paratope (antigen binding site) of an antibody of interest. More generally, U.S. Pat. No. 4,433,092 to Geysen (1989) describes a method of detecting or determining a sequence of monomers which is a topographical equivalent of a ligand which is complementary to the ligand binding site of a particular receptor of interest. Similarly, U.S. Pat. No. 5,480,971 discloses linear C₁₋₇-alkyl peralkylated oligopeptides and sets and libraries of such peptides, as well as methods for using such oligopeptide sets and libraries for determining the sequence of a peralkylated oligopeptide that preferentially binds to an acceptor molecule of interest. Thus, non-peptide analogs of the epitope-bearing peptides of the invention also can be made routinely by these methods.

As one of skill in the art will appreciate, polypeptides of the present invention and the epitope-bearing fragments thereof described above can be combined with parts of the constant domain of immunoglobulins (IgG), resulting in chimeric polypeptides. These fusion proteins facilitate purification and show an increased half-life in vivo. This has been shown, e.g., for chimeric proteins consisting of the first two domains of the human CD4-polypeptide and various domains of the constant regions of the heavy or light chains of mammalian immunoglobulins (EPA 394,827; Traunecker et al., Nature 331:84-86 (1988)). Fusion proteins that have a disulfide-linked dimeric structure due to the IgG part can also be more efficient in binding and neutralizing other molecules than a monomeric protein or protein fragment alone (Fountoulakis et al., J. Biochem. 270:3958-3964 (1995)).

### Diagnostic Methods

The present invention provides methods for diagnosing neurodegeneration in an individual by detecting the presence of, or determining levels of, biomarker polynucleotides (for example, mRNA) or biomarker polypeptides in a biological sample from the individual, including but not limited to samples that include neural cells (neurons or glial cells) from the individual. Such diagnostic methods are useful for diagnosis of diseases and conditions involving neurodegeneration including but not limited to Alzheimer's disease, Parkinson's disease, amyotrophic lateral sclerosis, Huntington's disease, and encephalitis and dementia resulting from infection with Human Immunodeficiency Virus. Such diagnostic methods are also useful for diagnosis or monitor of acute neural cell injuries such as brain ischemia and stroke and, trauma, and viral or prion infection.

In the methods of the present invention, a measurement of levels of a biomarker polypeptide or polynucleotide is compared to a “reference.” Depending on the embodiment of the invention, such a reference can include a measurement or ratio in a control sample; a standard value obtained by measurements of a population of individuals; a baseline value determined for the same individual at an earlier timepoint, e.g., before commencing a course of treatment; or any other suitable reference used for similar methods.

As used herein, the term “individual” or “patient” refers to a mammal, including, but not limited to, a mouse, rat, rabbit, cat, dog, monkey, ape, human, or other animal.

By “biological sample” is intended any biological sample obtained from an individual, including but not limited to, a neural cell (e.g., a neuron or glial cell), tissue, tissue culture, or other source that contains a biomarker protein or mRNA. Methods for obtaining such samples from mammals are well known in the art.

Detection of mRNA. Total cellular RNA can be isolated from a biological sample using any suitable technique such as the single-step guanidinium-thiocyanate-phenol-chloroform method described in Chomczynski and Sacchi, Anal. Biochem. 162:156-159 (1987). Levels of mRNA are then assayed using any appropriate method. These include Northern blot analysis, S1 nuclease mapping, the polymerase chain reaction (PCR), reverse transcription in combination with the polymerase chain reaction (RT-PCR), and reverse transcription in combination with the ligase chain reaction (RT-LCR).

Northern blot analysis can be performed as described in Harada et al., Cell 63:303-312, 1990). Briefly, total RNA is prepared from a biological sample as described above. For the Northern blot, the RNA is denatured in an appropriate buffer (such as glyoxal/dimethyl sulfoxide/sodium phosphate buffer), subjected to agarose gel electrophoresis, and transferred onto a nitrocellulose filter. After the RNAs have been linked to the filter by a UV linker, the filter is prehybridized in a solution containing formamide, SSC, Denhardt's solution, denatured salmon sperm, SDS, and sodium phosphate buffer. Then cDNA labeled according to any appropriate method (such as a ³²P-multiprimed DNA labeling system is used as probe. After hybridization overnight, the filter is washed and exposed to x-ray film. cDNA for use as probe according to the present invention is described in the sections above.

S1 mapping can be performed as described in Fujita et al., Cell 49:357-367, 1987). To prepare probe DNA for use in S1 mapping, the sense strand of above-described cDNA is used as a template to synthesize labeled antisense DNA. The antisense DNA can then be digested using an appropriate restriction endonuclease to generate further DNA probes of a desired length. Such antisense probes are useful for visualizing protected bands corresponding to the target mRNA. Northern blot analysis can be performed as described above.

According to one embodiment, levels of a particular biomarker mRNA is assayed using a polynucleotide amplification method, including but not limited to a polymerase chain reaction (PCR). One PCR method that is useful in the practice of the present invention is the RT-PCR method described in Makino et al., Technique 2:295-301, 1990), for example. By this method, the radioactivity of the DNA products of the amplification, i.e., the “amplification products” or “amplicons,” in the polyacrylamide gel bands is linearly related to the initial concentration of the target mRNA. Briefly, this method involves adding total RNA isolated from a biological sample in a reaction mixture containing a RT primer and appropriate buffer. After incubating for primer annealing, the mixture can be supplemented with a RT buffer, dNTPs, DTT, RNase inhibitor and reverse transcriptase. After incubation to achieve reverse transcription of the RNA, the RT products are then subject to PCR using labeled primers. Alternatively, rather than labeling the primers, a labeled dNTP can be included in the PCR reaction mixture. PCR amplification can be performed in a DNA thermal cycler according to conventional techniques. After a suitable number of rounds to achieve amplification, the PCR reaction mixture is electrophoresed on a polyacrylamide gel. After drying the gel, the radioactivity of the appropriate bands (corresponding to the mRNA) are quantified using an imaging analyzer. RT and PCR reaction ingredients and conditions, reagent and gel concentrations, and labeling methods are well known in the art.

According to one embodiment of an amplification method of the invention, primers are employed that selectively amplify a biomarker polynucleotide in a sample, for example, a primer pair including at least one primer that selectively hybridizes to biomarker mRNA (e.g., that includes sequences from the region of the biomarker mRNA that encodes a corresponding biomarker polypeptide. The second primer can include any sequence from the target biomarker polynucleotide. This embodiment is useful for amplifying only a biomarker transcript (mRNA) in a sample, for example.

According to another embodiment of the invention, primers are employed that selectively amplify a biomarker polynucleotide, for example, a primer pair that includes at least one primer that selectively hybridizes to biomarker mRNA (e.g., that includes sequences from exon 4a. The second primer can include any sequence from the target biomarker polynucleotide. This embodiment is useful for amplifying only a biomarker transcript (mRNA) in a sample, for example.

According to another embodiment of the invention, primers are employed that amplify both a biomarker polynucleotide and, for example, a control polynucleotide. For example, two primer pairs (i.e., 4 primers) can be used, one pair that selectively amplifies a biomarker polynucleotide and a second pair that selectively amplifies the control polynucleotide, so as to produce amplification products that can be distinguished from one another, for example by length.

The skilled artisan will be able to produce additional primers, primer pairs, and sets of primers for PCR and other amplification methods based on the sequences taught herein.

One embodiment of the present invention is a kit that includes primers useful for amplification methods according to the present invention. Such kits also include suitable packaging, instructions for use, or both.

Another PCR method useful for detecting the presence of and/or quantitating biomarker mRNA and protein in a biological sample, is through the use of “bio-barcode” nanoparticles. For detection and/or quantitation of proteins, for example, two types of capture particles are employed: one is a micro-size magnetic particle bearing an antibody selective for a target protein, and the other is a nanoparticle with attached antibodies selective for the same protein. The nanoparticle also carries a large number (e.g., ˜100) of unique, covalently attached oligonucleotides that are bound by hybridization to complementary oligonucleotides. The latter are the “bio-barcodes” that serve as markers for a selected protein. Because the nanoparticle probe carries many oligonucleotides per bound protein, there is substantial amplification, relative to protein. There is a second amplification of signal in a silver enhancement step. The result is 5-6 orders of magnitude greater sensitivity for proteins than ELISA-based assays, by detecting tens to hundreds of molecules. See, e.g., U.S. Pat. No. 6,974,669. See also, e.g., Stoeva et al., J. Am. Chem. Soc. 128:8378-8379, 2006, for an example of detection of protein cancer markers with bio-barcoded nanoparticle probes. The bio-barcode method can also be used for detecting and/or quantitating mRNA and other polynucleotides in a sample (Huber et al., Nucl. Acids Res. 32:e137, 2004; Cheng et al., Curr. Opin. Chem. Biol. 10:11-19, 2006; Thaxton et al., Clin. Chim. Acta 363:120-126, 2006; U.S. Pat. No. 6,974,669).

Detection of polypeptide. Assaying the presence of, or quantitating, biomarker polypeptide in a biological sample can occur using any art-known method.

Antibody-based techniques are useful for detecting the presence of and/or quantitating biomarker polypeptide levels in a biological sample. For example, expression of a biomarker polypeptide in tissues can be studied with classical immunohistological methods. In these, the specific recognition is provided by the primary antibody (polyclonal or monoclonal) but the secondary detection system can utilize fluorescent, enzyme, or other conjugated secondary antibodies. As a result, an immunohistological staining of tissue section for pathological examination is obtained. Tissues can also be extracted, e.g., with urea and neutral detergent, for the liberation of a biomarker polypeptide for Western-blot or dot/slot assay (Jalkanen et al., J. Cell. Biol. 101:976-985, 1985; Jalkanen et al., J. Cell. Biol. 105:3087-3096, 1987). In this technique, which is based on the use of cationic solid phases, quantitation of a particular biomarker polypeptide can be accomplished using isolated biomarker polypeptide as a standard. This technique can also be applied to body fluids. With these samples, a molar concentration of the particular biomarker polypeptide will aid to set standard values of biomarker polypeptide content for different tissues, fecal matter, body fluids (serum, plasma, urine, synovial fluid, spinal fluid), etc. The normal appearance of biomarker polypeptide amounts can then be set using values from healthy individuals, which can be compared to those obtained from a test subject.

Other antibody-based methods useful for detecting biomarker polypeptide levels include immunoassays, such as the enzyme linked immunosorbent assay (ELISA), the radioimmunoassay (RIA), and the “bio-barcode” assays described above. For example, monoclonal antibodies selective for a particular biomarker polypeptide can be used both as an immunoadsorbent and as an enzyme-labeled probe to detect and quantify the biomarker polypeptide. The amount of biomarker polypeptide present in the sample can be calculated by reference to the amount present in a standard preparation using a linear regression computer algorithm. Such an ELISA for detecting a tumor antigen is described in lacobelli et al., Breast Cancer Research and Treatment 11:19-30, 1988. In another ELISA assay, two distinct selective monoclonal antibodies can be used to detect biomarker polypeptide in a body fluid. In this assay, one of the antibodies is used as the immunoadsorbent and the other as the enzyme-labeled probe.

The above techniques may be conducted essentially as a “one-step” or “two-step” assay. The “one-step” assay involves contacting a sample containing a biomarker polypeptide with immobilized antibody and, without washing, contacting the mixture with the labeled antibody. The “two-step” assay involves washing before contacting the mixture with the labeled antibody. Other conventional methods may also be employed as suitable. It is usually desirable to immobilize one component of the assay system on a support, thereby allowing other components of the system to be brought into contact with the component and readily removed from the sample.

Suitable enzyme labels include, for example, those from the oxidase group, which catalyze the production of hydrogen peroxide by reacting with substrate. Glucose oxidase, for example, has good stability and its substrate (glucose) is readily available. Activity of an oxidase label may be assayed by measuring the concentration of hydrogen peroxide formed by the enzyme-labeled antibody/substrate reaction. Besides enzymes, other suitable labels include radioisotopes, such as iodine (¹²⁵I, ¹²¹I), carbon (¹⁴C), sulfur (³⁵S), tritium (³H), indium (¹¹²In), and technetium (⁹⁹Tc), and fluorescent labels, such as fluorescein and rhodamine, and biotin.

In addition to assaying biomarker polypeptide levels in a biological sample obtained from an individual, biomarker polypeptide can also be detected in vivo by imaging. Antibody labels or markers for in vivo imaging of biomarker polypeptide include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which may be incorporated into the antibody by labeling of nutrients for the relevant hybridoma.

An biomarker polypeptide-selective antibody or antibody fragment which has been labeled with an appropriate detectable imaging moiety, such as a radioisotope (for example, ¹³¹I, ¹¹²In, ⁹⁹mTc), a radio-opaque substance, or a material detectable by nuclear magnetic resonance, is introduced (for example, parenterally, subcutaneously or intraperitoneally) into the mammal to be examined for a disorder. It will be understood in the art that the size of the subject and the imaging system used will determine the quantity of imaging moieties needed to produce diagnostic images. In the case of a radioisotope moiety, for a human subject, the quantity of radioactivity injected will normally range from about 5 to 20 millicuries of ⁹⁹mTc. The labeled antibody or antibody fragment will then preferentially accumulate at the location of cells which contain a particular biomarker polypeptide. In vivo tumor imaging is described in Burchiel et al., “Immunopharmacokinetics of Radiolabeled Antibodies and Their Fragments” (Chapter 13 in Tumor Imaging: The Radiochemical Detection of Cancer, Burchiel and Rhodes, eds., Masson Publishing Inc., 1982).

Selective antibodies for use in the present invention can be raised against the intact biomarker polypeptide or an antigenic polypeptide fragment thereof, which may presented together with a carrier protein, such as an albumin, to an animal system (such as rabbit or mouse) or, if it is long enough (at least about 25 amino acids), without a carrier.

As used herein, the term “antibody” (Ab) or “monoclonal antibody” (Mab) is meant to include intact molecules as well as antibody fragments (or “fragment antibodies”) (such as, for example, Fab and F(ab′).sub.2 fragments) which are capable of selectively binding to a biomarker polypeptide. Fab and F(ab′).sub.2 fragments lack the Fc portion of intact antibody, clear more rapidly from the circulation, and may have less non-specific tissue binding of an intact antibody (Wahl et al., J. Nucl. Med. 24:316-325, 1983).

The antibodies of the present invention may be prepared by any of a variety of methods. For example, cells expressing the biomarker polypeptide or an antigenic fragment thereof can be administered to an animal in order to induce the production of sera containing polyclonal antibodies. In one method, a preparation of biomarker polypeptide is prepared and purified as described above to render it substantially free of natural contaminants. Such a preparation is then introduced into an animal in order to produce polyclonal antisera of greater specific activity.

The antibodies of the present invention include monoclonal antibodies (or binding fragments thereof). Such monoclonal antibodies can be prepared using hybridoma technology (Colligan, Current Protocols in Immunology, Wiley Interscience, New York (1990-1996); Harlow & Lane, Antibodies: A Laboratory Manual, Cold Spring Harbor Press, Cold Spring Harbor, N.Y. (1988), Chapters 6-9, Current Protocols in Molecular Biology, Ausubel, infra, Chapter 11). In general, such procedures involve immunizing an animal (for example, a mouse or rabbit) with an antigen or with an cell expressing a biomarker polypeptide. Suitable cells can be recognized by their capacity to bind anti-biomarker polypeptide antibody. Such cells may be cultured in any suitable tissue culture medium, such as Earle's modified Eagle's medium supplemented with 10% fetal bovine serum (inactivated at about 56° C.), and supplemented with about 10 μg/l of nonessential amino acids, about 1,000 U/ml of penicillin, and about 100 μg/ml of streptomycin. The splenocytes of such mice are extracted and fused with a suitable myeloma cell line. Any suitable myeloma cell line may be employed in accordance with the present invention. After fusion, the resulting hybridoma cells are selectively maintained in HAT medium, and then cloned by limiting dilution as described by Wands et al., Gastroenterology 80:225-232, 1981); Harlow & Lane, infra, Chapter 7. The hybridoma cells obtained through such a selection are then assayed to identify clones which secrete antibodies capable of binding the biomarker polypeptide antigen.

Alternatively, additional antibodies capable of binding to the biomarker polypeptide antigen may be produced in a two-step procedure through the use of anti-idiotypic antibodies. Such a method makes use of the fact that antibodies are themselves antigens, and therefore it is possible to obtain an antibody which binds to a second antibody. In accordance with this method, biomarker polypeptide-selective antibodies are used to immunize an animal, such as a mouse. The splenocytes of such an animal are then used to produce hybridoma cells, and the hybridoma cells are screened to identify clones which produce an antibody whose ability to bind to the selective antibody can be blocked by the antigen. Such antibodies comprise anti-idiotypic antibodies to the selective antibody and can be used to immunize an animal to induce formation of further selective antibodies.

It will be appreciated that Fab and F(ab′)₂ and other fragments of the antibodies of the present invention may be used according to the methods disclosed herein. Such fragments are typically produced by proteolytic cleavage, using enzymes such as papain (to produce Fab fragments) or pepsin (to produce F(ab′)₂ fragments). Alternatively, binding fragments can be produced through recombinant DNA technology or protein synthesis.

Where in vivo imaging is used to detect enhanced levels of a particular polypeptide for diagnosis in humans, one may use “humanized” chimeric monoclonal antibodies. Such antibodies can be produced using genetic constructs derived from hybridoma cells producing the monoclonal antibodies described above. Methods for producing chimeric antibodies are known in the art. See, for review, Morrison, Science 229:1202, 1985; Oi et al., BioTechniques 4:214, 1986; Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., Nature 312:643, 1984; Neuberger et al., Nature 314:268, 1985.

Further suitable labels for the selective antibodies of the present invention are provided below. Examples of suitable enzyme labels include malate dehydrogenase, staphylococcal nuclease, delta-5-steroid isomerase, yeast-alcohol dehydrogenase, alpha-glycerol phosphate dehydrogenase, triose phosphate isomerase, peroxidase, alkaline phosphatase, asparaginase, glucose oxidase, beta-galactosidase, ribonuclease, urease, catalase, glucose-6-phosphate dehydrogenase, glucoamylase, and acetylcholine esterase.

Examples of suitable radioisotopic labels include ³H, ¹¹¹In, ¹²⁵I, ¹³¹I, ³²P, ³⁵S, ¹⁴C, ⁵¹Cr, ⁵⁷To, ⁵⁸Co, ⁵⁹Fe, ⁷⁵Se, ¹⁵²Eu, ⁹⁰Y, ⁶⁷Cu, ²¹⁷Ci, ²¹¹At, ²¹²Pb, ⁴⁷Sc, ⁰⁹Pd, etc. ¹¹¹In has advantages where in vivo imaging is used since its avoids the problem of dehalogenation of the ¹²⁵I- or ¹³¹I-labeled monoclonal antibody by the liver. In addition, this radionucleotide has a more favorable gamma emission energy for imaging (Perkins et al., Eur. J. Nucl. Med. 10:296-301, 1985); Carasquillo et al., J. Nucl. Med. 28:281-287, 1987). For example, ¹¹¹In coupled to monoclonal antibodies with 1-(P-isothiocyanatobenzyl)-DPTA has shown little uptake in non-tumorous tissues, particularly the liver, and therefore enhances specificity of tumor localization (Esteban et al., J. Nucl. Med. 28:861-870, 1987).

Examples of suitable non-radioactive isotopic labels include ¹⁵⁷Gd, ⁵⁵Mn, ¹⁶²Dy, ⁵²Tr, and ⁵⁶Fe.

Examples of suitable fluorescent labels include ¹⁵²Eu label, fluorescein, isothiocyanate, rhodamine, phycoerythrin, phycocyanin, allophycocyanin, o-phthaldehyde, and fluorescamine.

Examples of suitable toxin labels include diphtheria toxin, ricin, and cholera toxin. Examples of chemiluminescent labels include luminal, isoluminal, aromatic acridinium ester, imidazole, acridinium salt, oxalate ester, luciferin, luciferase, and aequorin.

Examples of nuclear magnetic resonance contrasting agents include heavy metal nuclei such as Gd, Mn, and Fe.

Typical techniques for binding the above-described labels to antibodies are provided by Kennedy et al. (Clin. Chim. Acta 70:1-31, 1976), and Schurs et al. (Clin. Chim. Acta 81:1-40, 1977). Coupling techniques mentioned in the latter are the glutaraldehyde method, the periodate method, the dimaleimide method, the m-maleimidobenzyl-N-hydroxy-succinimide ester method.

One use for diagnostic compositions and methods of the present invention is for the detection of the presence of neurodegeneration (or another condition marked by the up-regulation and/or increased expression of a biomarker polypeptide) in an individual. Another use is to determine the presence and level of a biomarker mRNA or polypeptide in an individual in order to monitor the efficacy of therapy directed toward a neurodegenerative disease. The diagnostic compositions and method of the present invention are also useful for determining the efficacy of therapeutic agents for treatment and prophylaxis of a particular neurodegenerative disease.

Discovery of Small Molecules that Bind to Biomarker Polypeptides

The identification and availability of biomarker polynucleotides and their corresponding biomarker polypeptides enable the discovery of molecules, including but not limited to organic compounds, that bind specifically to (or are bound by) biomarker polypeptides and that can be labeled for use as probes for diagnostic purposes, among other uses. One aspect of the present invention, therefore, includes methods for discovering such small molecules using biomarker polypeptides.

According to such small molecule screening efforts, a sample is provided that comprises a selected biomarker polypeptide, such as a recombinant biomarker polypeptide. The sample is contacted with a chemical compound being tested, and it is determined whether the small molecule binds to (or is bound by) the biomarker polypeptide. Any well known method for assaying binding of a compound to a biomarker polypeptide may be used in such screening efforts, including in vivo, in vitro, and in silico methods. Depending on the properties of the biomarker polypeptide, different screening methods can be applied to screen combinatorial or natural chemical library. For example, if a biomarker is an enzyme, an assay can be established based on its ability to convert its substrate (for example, designed to show a difference in the concentration of the substrate before or after converting) in the presence or absence of a candidate chemical. Also, if a specific antibody is available, a protein-based screening assay for a combinatorial chemical library can be established based on, for example, the ability of a specific chemical to interfere with the binding of the biomarker polypeptide to its specific antibody conjugated with a fluorophore, for example. One can also establish a cell-based screen assay based on the same principle if the biomarker polypeptide expresses on the cell surface. Once a bound compound is found by these assays, it can serve as a lead compound for synthesizing better compounds by chemical methods such that desired chemical properties can be obtained in terms of the binding affinity, solubility, and membrane permeability. According to one embodiment of the invention, the compound crosses the blood-brain barrier to bind to the biomarker polypeptide in brain tissue that is affected by a neurodegenerative disease or condition. If labeled, the binding of the labeled compound (such as, for example, a radioligand-labeled compound) to the biomarker polypeptide can be detected by standard non-invasive methods such as positron emission tomography (PET) or single photon emission computed tomography (SPECT).

The invention will be better understood by reference to the following Examples, which are intended to merely illustrate the best mode now known for practicing the invention. The scope of the invention is not to be considered limited thereto.

### EXAMPLE 1

### Identification and Characterization of Biomarkers of Neurodegenerative Disease

We sought to identify genes whose expression is changed in the spinal cord of SOD1G93A mice using gene profiling, biochemical, and immunohistochemical methods. Surprisingly, we have shown that a number of interferon-stimulated genes (ISGs) are up-regulated specifically in astrocytes at a pre-symptomatic age, about 30 days before disease onset. In addition, the up-regulated ISGs are only observed in astrocytes surrounding motor neurons, implicating that they are most likely induced by pathological events in motor neurons. Furthermore, cultured astrocytes are highly sensitive to interferon, especially type I interferon, and the resulting ISGs are independent of genes implicated in classical gliosis. All these results suggest that the activation of IFN signaling pathway in SOD1 spinal cord may represent an early “dialogue” between motor neurons and glial cells in response to SOD 1 mutant-induced toxicity.

### Methods and Materials

Animals. SOD1(G93A) transgenic mice, which express the human SOD1 gene containing the G93A mutation [the B6SJL-TgN(SOD1-G93A)1Gur line, Jackson Laboratory, Bar Harbor, Me., USA], were maintained as hemizygotes by breeding transgenic males with wildtype B6SJL females. The animals were genotyped for expression of the transgene by the polymerase chain reaction (PCR) according to a protocol from the Jackson Laboratory. The mice were housed in a virus-free barrier facility under a 12-hour (h) light/dark cycle, with ad libitum access to food and water.

Cell culture. Primary spinal cord astrocyte cultures were obtained from P₀-P₁ C57/BL6 mouse pups. Spinal cords were dissected from cervical to lumbar regions in filter-sterilized Hank's balanced salt solution (HBSS) (Invitrogen) containing 25 mM glucose. Then tissue was stripped of meninges and blood vessels, minced, and incubated for 20 min at 37° C. and 95% O₂/5% CO₂ in HBSS plus 0.5 mM EDTA and 0.5% trypsin. The enzyme solution was aspirated, and the tissue was rinsed with culture medium containing DMEM (Life Technologies, Grand Island, N.Y.), 2 mM glutamine, 10% fetal bovine serum (FBS; HyClone, Logan, Utah), and 100 U/ml of penicillin/streptomycin. Tissue was dissociated by trituration (20×) with a fire-polished Pasteur pipette. Dissociated cells were plated on poly-L-lysine-coated 75 cm² flasks and maintained at 37° C. and 95% O₂/5% CO₂ in culture medium. The medium was changed every 3-4 days. Ten days later, cultures were shaken at 37° C., 250 rpm for 24 h. After rinsed with culture medium, the cells were passed from the flasks into Petri dishes with or without glass slides. The secondary cultures were grown for ≧8 days in vitro (DIV) before being treated with IFNβ, IFNγ, LPS, IL1β, or TNFα.

Gene expression array analysis. Total RNA was extracted from the total homogenates of the lumbar spinal cords from three P60 female SOD1 mice and their wildtype female littermates using RNeasy (Qiagen). A DNA removal step was included in all RNA extraction experiments. Total RNA was then used as a template for double-stranded cDNA synthesis (MessegeAMP II kit, Ambion), which was used as a template for biotin-labeled cRNA synthesis (High Yield RNA Transcription Labeling kit; Affymetrix). Biotin-labeled cRNA samples were submitted to the DNA Microarray Core facility at the University of California, Los Angeles, for hybridization and scanning by use of a standardized protocol. Commercial oligonucleotide microarray chips (Mouse Genome 430 2.0 Array) were obtained from Affymetrix. Six chips were hybridized, each corresponding to one mouse. Microarray Suite 5.0 was used to generate CEL files using the default settings. dChip software (Li and Wong, Genome Biol 2: Research0032.0031-0032.0011, 2001) was used for calculation of expression levels because it has been shown to operate consistently well in comparison to other analysis software/algorithms (Zakharkin et al., BMC Bioinformatics 6: 214-214, 2005). Genes differentially expressed in the SOD1 spinal cord compared to wildtype spinal cord were selected by using comparison analysis in dChip.

Real-time PCR analysis. Total RNA extracted from lumbar spinal cords or spinal ventral grey matter was used as a template for cDNA synthesis by using Superscript II (Invitrogen, Carlsbad, Calif., USA) (Xing et al., Eur J Neurosci 24:2987-92, 2006). Quantitative real-time PCR was performed with the Stratagene Mx3000p QPCR system (Stratagene, La Jolla, Calif., USA) using SYBR Green PCR Master Mix (Applied Biosystems, Foster City, Calif., USA). Each sample was analyzed in triplicate. At the end of the PCR, baseline and threshold values were set in the software and the cycle threshold values were exported to Microsoft Excel for analysis. Gene expression was normalized to glyceraldehyde-3-phosphate dehydrogenase (GAPDH) to correct for RNA input variability. The primers designed using the Primer3 software (http://www-genome.wi.mit.edu/cgi-bin/primer/primer3_www.cgi) were: Gbp2 5′-tgtagaccaaaagttccagacaga-3′ (forward), 5′-gataaaggcatctcgcttgg-3′ (reverse); Ifi27, 5′-tagccacactccaatcagca-3′ (forward), 5′-agagcaaggctccaacagc-3′ (reverse); Ifi44, 5′-ctgattacaaaagaagacatgacagac-3′ (forward), 5′-aggcaaaaccaaagactcca-3′ (reverse); Ifit1, 5′-ggacaaggtggagaaggtgt-3′ (forward), 5′-tcctcacagtccatctcagc-3′ (reverse); Ifit-3,5′-ggaatgcccagaacttgaat-3′ (forward), 5′-gctcgttcatttcttccaca-3′ (reverse); Isg15, 5′-gaacaagtccacgaagaccag-3′ (forward), 5′-gcagctccttgtcctccat-3′ (reverse); Oas1a, 5′-gctgccagcctttgatgt-3′ (forward), 5′-tggcatagattctgggatca-3′ (reverse); Usp18, 5′-ggcagtgcttaggtgacaga-3′ (forward), 5′-gagagatcccatgaaccgat-3′ (reverse); and GAPDH, 5′-ggcattgctctcaatgacaa-3′ (forward), 5′-tgtgagggagatgctcagtg-3′ (reverse).

Western blot analysis. To perform the western blot analysis, the lumbar spinal cord of SOD1 mice and their wildtype littermates, or the pellet of primary spinal cord astrocyte cultures, were homogenized in SDS-sample buffer and the proteins were fractionated by SDS-PAGE and transferred to Hybond ECL nitrocellulose membranes (Amersham Pharmacia Biotech) in Tris-glycine buffer. The blots were probed with rabbit anti-Isg15 primary antibody (1:3000 dilution), rabbit anti-Usp18 antibody (1:500 dilution), mouse anti-β actin antibody (1:5000 dilution, Pierce), mouse anti-Gfap antibody (1:5000 dilution, Sigma), rabbit anti-phospho-Stat1 (Tyr701) antibody (1:1000, Cell Signaling), or rabbit anti-phospho-Stat2 (Tyr689) antibody (1:1000, Upstate). The blots were subsequently probed with either anti-rabbit or anti-mouse IgG peroxidase conjugate (Amersham), and the signals were detected using ECL reagents (Amersham).

Immunohistochemistry. SOD1 mice and their wildtype littermates at P45-P100 were anesthetized with isoflurane and perfused transcardially with chilled PBS followed by phosphate-buffered 4% paraformaldehyde solution. The spinal cord was dissected immediately, postfixed for 2 hours, and cytoprotected in 25% sucrose overnight. Transverse cryostat sections (12 μm) were processed for immunohistochemistry as described previously (Wang and Zhang, Eur. J. Neurosci. 22:2376-2380, 2005). In brief, sections were incubated at room temperature (RT) with rabbit anti-Isg 5 (1:1000) for 1 h, followed by incubation with a biotinylated goat anti-rabbit antibody (1:200, Vector Laboratories, Burlingame, Calif., USA). The detection of Isg 15 immunoreactivity was performed using a Vectastain Elite ABC kit (Vector Laboratories) and visualized with a DAB substrate solution (Roche Applied Science, Penzberg, Germany). For immunofluorescence, spinal cord sections were incubated at room temperature (RT) with rabbit anti-Isg15 (1:1000), mouse anti-Gfap (1:1000, Sigma), mouse anti-NeuN (1:1000, Millipore), or rat anti-F4/80 (1:100, Pharmingen), followed by incubation with Alexa Fluor 555 anti-rabbit IgG (Invitrogen), Alexa Fluor 488 anti-rabbit IgG (Invitrogen), FITC anti-mouse IgG (Jackson Immunoresearch), Alexa Fluor 555 anti-mouse IgG (Invitrogen), RRX anti-rat IgG (Jackson Immunoresearch). Some sections were also incubated with FITC lectin (from tomato) (Vector Laboratories). After washing in PBS 3-5 times, the samples were mounted by adding Vectashield (Vector Laboratories) to the cover glasses.

### Results

We used a gene profiling approach to screen the early molecular changes in the spinal cord of pre-symptomatic SOD1 mice.

IFN-stimulated genes in the spinal cord of pre-symptomatic SOD1 mouse. RNA was isolated from the lumbar spinal cords of 2-month-old female SOD1 mice and their wild-type littermates. After linear RNA amplification, the target RNA was hybridized to Affymetrix murine genome oligonucleotide DNA array 430 2.0. Raw data were processed in dChip and model-based expression values were calculated by perfect match-only method. Probe sets with an average expression value of 30 in either SOD1 mice or wild-type controls were first filtered, and then differentially regulated genes were selected by comparison analysis using a lower 90% confidence bound fold change of 1.2 (t-test, p<0.05). Gene-annotation analysis was performed by using the Database for Annotation, Visualization and Integrated Discovery (DAVID) 2007 (http://david.abcc.ncifcrf.gov/). Surprisingly, closer examination of the data showed that a number of IFN-stimulated genes (ISGs) were significantly up-regulated in the SOD1 spinal cords (Table 2) (Der et al., Proc. Natl. Acad. Sci. USA 95:15623-15628, 1998). The fold changes of SOD1/WT were from 1.28 to 2.76. Some ISGs were preferentially induced by type I IFN, such as Ifi27, Ifi44, Ifit1, Ifit2, Isg15, Oas1a, and Usp18. Some were preferentially induced by type II IFN, such as Cxcl10, Gbp2, Iitp, Iigp1, Iigp2, and Psmb8. STAT1, a transcription factor involved in both type I and type II IFN signaling, and Irf8 and Isgf3g, transcription factors specifically involved in type I IFN signaling, were also significantly up-regulated.

TABLE 2
IFN-stimulated genes identified in the spinal cord of pre-symptomatic
SOD1 mice. Fold change is the ratio of expression in SOD1
samples/expression in wild-type controls. P value is from student t test.

| | RefSeq | Fold | |
|---|---|---|---|
| Genes | Accession # | change | P value |
| Usp18: ubiquitin specific peptidase 18 | NM_011909 | 2.76 | 0.0090 |
| | NM_008327 | | |
| Ifi202: interferon activated gene 202 | NM_011940 | 2.71 | 0.0325 |
| Iigp1: interferon inducible GTPase 1 | NM_021792 | 2.64 | 0.0208 |
| Ifi27: interferon, alpha-inducible | NM_029803 | 2.39 | 0.0001 |
| protein 27 | | | |
| Ifit3: interferon-induced protein with | NM_010501 | 2.35 | 0.0095 |
| tetratricopeptide repeats 3 | | | |
| Oasl2: 2′-5′ oligoadenylate synthetase- | NM_011854 | 2.22 | 0.0013 |
| like 2 | | | |
| Ifit1: interferon-induced protein with | NM_008331 | 2.21 | 0.0023 |
| tetratricopeptide repeats 1 | | | |
| Rsad2: radical S-adenosyl methionine | NM_021384 | 2.13 | 0.0372 |
| domain containing 2 | | | |
| Ifi44: interferon-induced protein 44 | NM_133871 | 2.08 | 0.0105 |
| Isg15: interferon, alpha-inducible | NM_015783 | 2.01 | 0.0052 |
| protein | | | |
| Cxcl10: chemokine (C—X—C motif) | NM_021274 | 1.89 | 0.0400 |
| ligand 10 | | | |
| Gbp2: guanylate nucleotide binding | NM_010260 | 1.73 | 0.0017 |
| protein 2 | | | |
| Socs3: suppressor of cytokine | NM_007707 | 1.60 | 0.0189 |
| signaling 3 | | | |
| Irf8: interferon regulatory factor 8 | NM_008320 | 1.57 | 0.0019 |
| Oas1a: 2′-5′ oligoadenylate synthetase | NM_145211 | 1.51 | 0.0241 |
| 1A | | | |
| Irgm: immunity-related GTPase | NM_008326 | 1.46 | 0.0110 |
| family, M | | | |
| B2m: beta-2 microglobulin | NM_009735 | 1.41 | 0.0124 |
| Psmb8: proteosome (prosome, | NM_010724 | 1.40 | 0.0132 |
| macropain) subunit, beta type 8 (large | | | |
| multifunctional peptidase 7) | | | |
| Igtp: interferon gamma induced | NM_018738 | 1.40 | 0.0424 |
| GTPase | | | |
| Isgf3g: interferon dependent positive | NM_008394 | 1.38 | 0.0026 |
| acting transcription factor 3 gamma | | | |
| Ifitm3: interferon induced | NM_025378 | 1.36 | 0.0162 |
| transmembrane protein 3 | | | |
| Stat1: signal transducer and activator | NM_009283 | 1.38 | 0.0010 |
| of transcription 1 | | | |
| Ifih1: interferon induced with helicase | NM_027835 | 1.32 | 0.0066 |
| C domain 1 | | | |
| Iigp2: interferon inducible GTPase 2 | NM_019440 | 1.31 | 0.0066 |
| Ifit2: interferon-induced protein with | NM_008332 | 1.28 | 0.0338 |
| tetratricopeptide repeats 2 | | | |
| Samhd1: SAM domain and HD | NM_018851 | 1.43 | 0.0398 |
| domain, 1 | | | |
| Clec7a: C-type lectin domain family 7, | NM_020008 | 2.93 | 0.0114 |
| member a | | | |

Validation of genes up-regulated in pre-symptomatic SOD1 mice. As most observed ISGs have not been reported previously in studies of SOD1 mice, we selected eight candidate ISGs including Gbp2, Ifi27, Ifi44, Ifit1, Ifit3, Isg15, Osa1a, and Usp18 for independent validation by quantitative RT-PCR (Q RT-PCR).

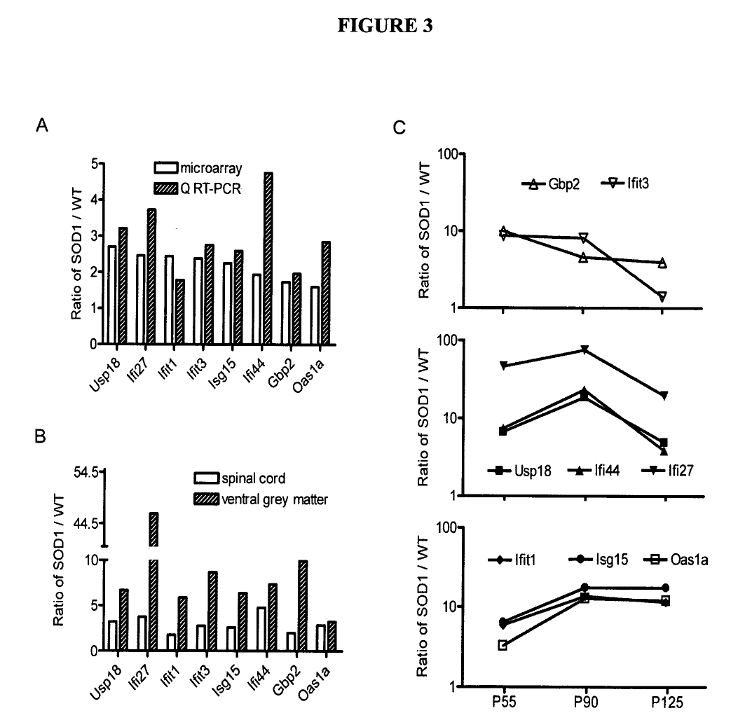

Total RNA was isolated from independent SOD1 lumbar spinal cords and wild-type controls. For each of the candidate genes a segment within the open reading frame was amplified using specific sets of primers. As a control, we amplified the housekeeping gene GAPDH, which had consistent expression levels in all arrays. Overall, the Q RT-PCR data showed a good concordance with the microarray results (FIG. 3A). We detected significantly higher levels of mRNA of all eight candidate genes in the total spinal cord RNA from SOD1 mice than from wild-type littermate controls with the ratios of SOD1/WT from 1.78 to 4.75. Since ALS-vulnerable motor neurons reside in the ventral horn of the spinal cord, we have also examined the expression levels of the candidate genes in the ventral grey matter. Interestingly, seven candidate genes Gbp2, Ifi27, Ifi44, Ifit1, Ifit3, Isg15, and Usp18 showed significantly higher levels of their mRNAs in the ventral grey matter than in the whole spinal cord (FIG. 3B), demonstrating that the expression pattern of these genes are region-specific in the spinal cord of the SOD1 mouse. We also found that the mRNA levels for individual genes varied extensively in the ventral grey matter at different disease stages (FIG. 3C). Gbp2 and Ifit3 had highest expression levels at the pre-symptomatic age of P55. Ifi27, Ifi44 and Usp18 had highest expression levels at P90, which is around disease onset. Ifit1, Isg15 and Oas1a expressed most at the late stage of the disease.

We next examined the protein levels of two candidate genes, Isg 15 and Usp18, in the lysates of the lumbar spinal cords from the SOD1 mice showing the sign of paresis. We found that both proteins had elevated expression levels in the SOD1 compared with wild-type lysates. We then investigated the temporal and spatial pattern of Isg15 expression in the spinal cord of SOD1 mice and their wild-type littermates by immunohistochemistry at different stages of the disease. We found that Isg15 immunoreactivity was very weak in the spinal cords of all wild-type mice and young SOD1 mice. From a pre-symptomatic age (P60) to an age following disease onset (P100), strong Isg15 signals appeared consistently in the ventral horns of the SOD1 spinal cords, where the ALS-vulnerable motor neurons reside. In the late stage of the disease, the induction of Isg15 was extended to other grey matter regions of the SOD1 spinal cord.

To determine which cell type expressed Isg15 in the SOD1 spinal cord, we performed double-immunostaining with a variety of cell type markers. We found that Isg15 was only co-localized with GFAP, an astrocyte marker. Isg15 was not co-localized with a neuronal marker NeuN, a microglia marker 14/80, or an endothelial cell/microglia marker lectin (from tomato). This result was also consistent with the observed size and morphology of Isg15-positive cells.

Phosphorylation of STAT1 and STAT2 in the spinal cord of pre-symptomatic SOD1 mice. It is well documented that activation of IFN signaling pathways leads to the phosphorylation of STAT1 and STAT2 transcription factors, and the translocation of phosphorylated STAT1 and STAT2 to nuclei plays an essential role in the induction of ISGs. To further confirm the activation of IFN signaling pathways in the SOD1 spinal cord, we determined the signal of phosphorylated STAT1 and STAT2 using immunohistochemistry with antibodies specifically against phosphorylated STAT1 and STAT2. Spinal cord sections of P50 and P80 SOD1 mice and their wild-type littermates were examined. We found that strong nuclear-like labeling of both phospho-STAT1 and phospho-STAT2 obviously appeared in the ventral horns of all SOD1 sections and that P80 SOD1 sections had more immunoreactive signals than that of P50. In contrast, nuclear-like labeling of phospho-STAT1 and phospho-STAT2 was not detected in the wild-type sections. The small size of the nuclear-like labeling (estimated at 3-5 μm) was also consistent with the size of astrocyte nuclei. Together with microarray data, these results suggest that IFN signaling pathways are activated in the spinal cord of SOD1 mouse at pre-symptomatic stage of the disease.

Type I IFN signaling pathway in the astrocytes of spinal cord. To further confirm that the induction of IFN-stimulated genes in the astrocytes of spinal cord is mediated by type I IFN signaling pathway, we have tested the primary astrocyte cultures of the spinal cord with IFNβ treatment. We found that IFN beta treatment induced a dramatic elevation of Isg15 in cultured astrocytes. Furthermore, the effect of IFNβ showed a time- and concentration-dependent manner. Most surprisingly, the induction of Isg15 in astrocytes was very sensitive to IFNβ: even a very low concentration of 1 unit/ml was enough to induce an obvious increase of Isg15. Elevation of Isg15 could be detected from two hours after the treatment of IFN beta. Another IFN-stimulated gene Usp18 was also increased from two hours after IFN beta treatment. In contrast, both phospho-STAT1 and phospho-STAT2 were increased to their highest levels within a half hour after IFN beta treatment, demonstrating phosphorylation of STAT1 and STAT2 occurred before the expression of ISGs. Six hours later, their levels in IFN beta treated astrocytes were still higher than that in untreated controls. The elevation of phospho-STAT1 and phospho-STAT2 totally disappeared fourteen hours after IFN-beta treatment. These results indicate the activation of the type I interferon pathway in astrocytes is a highly sensitive and dynamic process. Interestingly, the expression of a gliosis marker, GFAP, was not altered by IFNβ treatment under the same condition, indicating a separate signaling pathway for gliosis.

We also treated the primary spinal cord astrocyte cultures with other cytokines including IFNγ (100 unit/ml), IL1β (10 ng/ml), TNFα (20 ng/ml), and LPS (1 μg/ml). All tested cytokines increased the expression of Isg15, although at a less degree than IFNβ did. Like IFNβ LPS also caused a very strong elevation of Isg15 expression. Interestingly, LPS-induced Isg15 expression was almost completely blocked by an anti-IFNβ antibody but not by an anti-IFNγ antibody. An anti-IFNα antibody had no effect on LPS-induced Isg15 expression either. These results suggest that IFNβ is a major trigger of ISGs expression in spinal cord astrocytes under the LPS-induced inflammatory condition.

### Discussion

We have used oligonucleotide arrays to study the changes of mRNA expression in the spinal cord of the SOD1 mice and have identified for the first time a number of IFN-stimulated genes up-regulated at the early pre-symptomatic stage (P60) of these mice. Some ISGs are preferentially induced by type I IFNs, including Ifi27, Ifi44, Ifit1, Ifit2, Isg15, Isgf3g, Oas1a, and Usp18. Some are preferentially induced by type II IFNg, including Cxcl10, Iitp, Iigp1, Iigp2, and Psmb8. It is well-known that type I and type II IFNs initiate two similar but distinct signaling pathways. Type I IFNs bind to their heterodimeric receptor and trigger phosphorylation of JAK1 and TYK2 tyrosine kinases, which in turn phosphorylated STAT1 and STAT2 transcription factors. Phospho-STAT1 and phospho-STAT2 move into the nucleus and form a complex with Isgf3g, which activates the transcription of many IFN-stimulated genes. Whereas type II IFNg acts on its tetrameric receptor and triggers JAK1 and JAK2 kinases, which subsequently phosphorylates STAT1. The phospho-STAT1 forms a homodimer, translocates to the nucleus, and induces the expression of IFNg-stimulated genes. The detection of both phospho-STAT1 and phospho-STAT2 in the spinal cord of pre-symptomatic SOD1 mice and in cultured astrocytes induced by interferon beta provides further evidence for the activation of interferon signaling pathways, especially type I interferon pathway. Activation of IFN signaling pathways in brain is usually associated with inflammation triggered by viral infection or autoimmunization. As SOD 1 mice show little sign of inflammation at early pre-symptomatic stage, the sensitive and robust activation of IFN signaling pathways at this stage of the disease is a surprising but intriguing finding.

We have also shown that most ISGs have transcribed much higher mRNA levels in the pre-symptomatic ventral horn than the whole spinal cord. This region-preferential expression pattern could also be detected at protein level. For example, the Isg15 protein was robustly induced only in the ventral horn of the SOD1 spinal cord as early as P60. Thus, the induction of ISGs is a pre-symptomatic molecular event occurring specifically in the region where the selectively vulnerable motor neurons reside. The temporal and spatial expression pattern of ISGs in the SOD1 mice suggests that the activation of IFN signaling pathways is triggered most likely by pathological changes in motor neurons. To further elucidate the molecular events in motor neurons that trigger the activation of IFN signaling pathway will be important to understand what occur in the early stage of ALS. Furthermore, these ISGs may be reliable and robust biomarkers for detecting early pathological changes in the spinal cord of ALS.

It has been reported that astrocytes may play a crucial role in the specific degeneration of spinal motor neurons in ALS. Our study showed that the astrocyte was the major cell type in the spinal cord to express ISGs upon stimulation. Double immunostaining experiments have showed that Isg15 was selectively co-localized with GFAP, an astrocyte marker, in the ventral horn of the SOD1 spinal cord. In addition, the expression of Isg15 in astrocytes was through type I IFN signaling pathway since its expression in the spinal cord was completely abolished in SOD1 mice at IFNAR1 null background. Furthermore, the expression of Isg15 could also be strongly induced by IFN beta, a type I interferon, in primary cultures of spinal cord astrocytes. Phosphorylation of STAT1 and STAT2, two major components of type I IFN signaling pathway, was observed prior to the increase of Isg15. The effective concentration of IFN beta could be as low as 1 unit per ml, suggesting that spinal cord astrocytes are very sensitive to type I IFN stimulation. IFN beta at all concentrations tested could not induce the expression of Isg15 in the neurons of spinal cord primary cultures. Our data suggest that the activation of type I IFN signaling pathway occurs specifically in astrocytes.

Most ISGs found in the present study are not well investigated as yet; only a minority of these genes have been studied in mainly immune system. Isg 15 is an ubiquitin like modifier and can protect against Sindbis virus-induced lethality (Lenschow et al., J. Virol. 79:13974-13983, 2005). Usp18 is a major Isg15-specific protease which specifically removes Isg15 from conjugated proteins (Malakhov et al., J. Biol. Chem. 277:9976-9981, 2002), and it is a novel in vivo inhibitor of signal transduction pathways that are specifically triggered by type I IFNs (Malakhova et al., EMBO J. 25:2358-2367, 2006). Transcriptional activation of Ifi202 gene may inhibit cell growth and increase cell survival under certain stress conditions (Xin et al., Oncogene 22:4831-4840, 2003). Enforced neuronal Iif27 expression results in a significant delay in Sindbis virus-induced death in neonatal mice (Labrada et al., J. Virol. 76:11688-11703, 2002). Gbp2 confers resistance to paclitaxel-induced cytotoxicity in tumor cells (Balasubramanian et al., Cell. Mol. Biol. [Noisy-le-grand] 52:43-49, 2006). Cxcl10 has been strongly implicated in the pathology of many CNS disorders, and therapeutic neutralization of Cxc10 decreases secondary degeneration and functional deficit after spinal cord injury in mice (Gonzalez et al., Regen. Med. 2:771-783, 2007). Expression of ISGs may result in different modification of cellular functions in the spinal cord of SOD1 mice, although there has been no report on the biological functions of ISGs in the ALS. Thus, our findings open a completely new avenue for investigating the role of these ISGs in ALS.

Given its ability to mark disease progression in SOD1 mice, we analyzed ISG15 expression in a second neuronal injury model. For this we chose the mouse stroke model employing relatively mild 30 minute bilateral common carotid artery (BCCA) occlusion, which is particularly effective in C57Black/6 mouse strains (Fuji et al., Stroke 28:180-5-1810, discussion 1811, 1997). Five days after the start of reperfusion, we examined expression of GFAP, Iba-1 and ISG15 in control and treated brain. The stroke-induced injury caused activation of astrocytes (labeled by GFAP) and microglia (labeled by Iba-1) in many brain regions five days after BCCA occlusion. While up-regulation of GFAP and Iba-1, marking gliosis, was more diffuse in whole brain, expression of ISG15 was restricted to the hippocampus, where neuronal degeneration was most severe. Low background staining in normal brain and robust expression in affected areas provided a greater signal/noise ratio and spatial resolution than that seen with GFAP and Iba-1, suggesting that ISG15 is a superior early biomarker predictive of neuronal injury, one particularly applicable to in vivo imaging. These results also demonstrated that ISG155 expression is triggered by neuronal injury and localized by the site of injury.

We have also tested the expression of Isg15 in type 1 interferon receptor knock-out mice, using the same ischemia model (BCCA occlusion). The induction of Isg15 by ischemia, as shown previously in normal mice, was no longer observed in this knock-out mouse line; even the ischemia-induced injury was evident by increasing expression of gliosis markers such as GFAP and Iba-1. This result suggests that the induction of interferon-stimulated genes observed in SOD1 mice is mediated by type 1 interferon receptors, which are mainly activated by interferon alpha and beta.

In summary, using microarray and Q-PCR methods we identified a group of interferon-stimulated genes that are up-regulated in pre-symptomatic stages of SOD1 mice, resulting specific up-regulation of IFN-stimulated genes in astrocytes near motor neurons of the spinal cord. Further investigation using immunohistochemistry revealed that one of these genes, ISG15, showed a robust response in both SOD1 and stroke models. The striking temporal and spatial expression patterns seen in the disease state and low background in normal mice suggest that these molecules are ideal biomarkers for ALS and other neurodegenerative diseases.

### EXAMPLE 2

### Expression of Usp18 in a Mouse Stroke Model

We also investigated the expression of another member of this gene group, Usp18, at the protein level. First, we tested the expression level of Usp18 in an Usp18 knock-out/LacZ knock-in mouse (Usp18^{LaZ}) after bilateral common carotid arteries (BCCA) occlusion, measured as the intensity of β-gal staining. Five days after occlusion, β-galactosidase staining was increased dramatically in the brain area affected by ischemia insult including striatum, thalamus, and hippocampus, but not in the neocortex, which was relatively spared. The control mouse only showed background staining in blood vessels and near central canal (FIG. 2, left panel). Second, we examined the expression level of Isg15, Isg15 conjugates, and Usp18 in the spinal cord of SOD 1 mice and their wild type controls by Western blot analysis. The expression levels of these proteins were all increased in the SOD1 mice compared with wild type mice, confirming and extending previous results obtained by immunohistochemistry.

### EXAMPLE 3

### Expression Pattern and Levels of Biomarkers During Disease Progression

The expression levels and patterns of a group of candidate genes in a mouse model of ALS, SOD1 mutant mice are determined, and expression of these genes is compared in terms of specificity, sensitivity (early detection) and robustness (high signal to noise ratio) in responses to neuronal injury.

Most motor neuron diseases show relatively slow onset and selective motor neuron death. Thus the expression of disease-related genes is expected to occur in motor neurons and neighboring glial cells during the disease process. Therefore, expression products of disease-related genes can serve as specific biomarkers underlying motor neuron death. In order to select ideal biomarkers, we compare the expression of a group of genes identified by our previous microarray experiments in order to (1) categorize genes according to sensitivity, specificity, and robustness in relation to disease progression in SOD1 mutant mice, and (2) identify biomarkers with diverse biochemical structures so that different techniques can be exploited for their detection. This facilitates the design of non-invasive methods to detect biomarkers, such as in vivo imaging.

We use independent methods to validate disease-related genes identified using microarrays to identify false positives and negatives. In addition, we analyze their temporal and spatial expression patterns to determine if their gene products are good markers of disease progression. Furthermore, we quantify the expression level of a given gene to assess its robustness in a given time period. We employ in situ hybridization, Q-PCR, immunohistochemistry, and Western blot analysis in SOD1 mutant mice to accomplish these goals.

Animal selection. We derive spinal cord samples from the most commonly used transgenic mouse model of ALS, the SOD1 (G93A) [strain name: TgN (SOD10G93A)1Gur] (Gurney et al., Science 264:1772-1775, 1994; available from Jackson Laboratory, Bar Harbor, Me.) and wild-type littermates. The SOD1 transgenic mouse line has a B6 genetic background. SOD1 (G93A) mutant mice show disease symptoms at about 90 days and die around 130 days. Therefore, we take spinal cord samples from P40 (pre-symptomatic) to P120 (paresis) at 10-day intervals.

Temporal and spatial expression patterns of motor neuron disease-related genes. We use in situ hybridization, Q-PCR, immunohistochemistry, and Western blot analysis to determine expression patterns and levels of disease-related genes in SOD1 mutant mice. Based on results from microarray experiments, we select nine candidate genes for this experiment (as listed in Table 2). We first analyze expression levels of these genes by Q-PCR to confirm microarray results and further investigate how robust expression is at additional time points. We then undertake in situ hybridization to analyze the spatial expression pattern of selected genes, whose expression levels were confirmed by Q-PCR, primarily in 40, 60 (pre-symptom), 90 (disease onset), and 120 (end stage) day-old SOD1 mutant mice and respective wild-type controls. Antibodies specific for proteins encoded by these genes are used in immunohistochemistry to analyze temporal and spatial expression of selected genes at the protein level and compare results with in situ hybridization and Q-PCR data. To complement the immunohistochemistry, we also use Western blot analysis to quantify expression levels of biomarkers. Finally, we identify cell types expressing these genes in the disease state using either laser capture microdissection (LCM) combined with Q-PCR or double immunostaining.

Animal breeding and symptom test. We breed SOD1 mutant mice to obtain hemizygotes. Mice are genotyped by PCR after weaning using protocols. We determine different disease stages based on the following criteria: (1) no sign of weakness (pre-symptomatic); (2) tremor and loss of splay reflex (disease onset); (3) weakness of one or both hindlimbs (paresis); and (4) paralysis of one or both hindlimbs (end stage). We will collect tissues from animals at similar disease stages (stages 1 to 3) at designated ages.

Quantitative PCR. We employ a Q-PCR protocol used previously in our laboratory (Xing, et al., Eur. J. Neurosci. 24: 2987-2992, 2006). Briefly, mice are anesthetized with isofluorane and then transcardially perfused with saline. The ventral grey matter of the spinal cord is isolated from wild-type control and SOD1 mutant mice as follows. Total RNA obtained from the sample is reverse transcribed into cDNA with a reverse transcription (RT)-PCR kit (Invitrogen, Carlsbad, Calif.). RNA (1-100 ng) is added to reverse transcription buffer containing 20 mM Tris-HCl, 50 mM KCL, 5 mM MgCl₂, 10 mM DTT, 0.5 mM dNTP mix, 1 μL RNAse inhibitor, 25 ng/L random hexamers and 50 units of SuperScript II reverse transcriptase. The sample is then incubated at 42° C. for 1 hour and the reaction terminated at 70° C. for 15 min. First strand cDNA is treated with 1 μL RNase H at 37° C. for 20 min to remove RNA.

cDNA from each sample is used in Q-PCR to quantify expression of genes selected from gene profiling experiments. PCR primers are designed using a program available on the internet (Primer 3, http://www-genome.wi.mit.edu/cgi-bin/primer/primer3_www.cgi). We use a LightCycler instrument (Roche Diagnostics Corporation, Indianapolis, Ind.) for real-time PCR experiments. The LightCycler uses SYBR Green I, a DNA double-strand-specific dye, to monitor progress of each PCR amplification by detecting SYBR Green I fluorescence. We use a 10- to 100-fold dilution of the first strand cDNA sample as a template for PCR. The PCR mix (20 μL total volume) contains 2 μL 10×RNA Master Mix from Roche (with buffer, SYBR Green I, nucleotides, and enzyme), additional MgCl₂ pre-tested by regular PCR for each gene, 2 μL of cDNA, and PCR-grade water. Forty cycles of PCR are undertaken using the following typical reaction conditions: 95° C., 2 s; 55-65° C., 5 s; and 72° C., 10-20 s. Products of each sample are subjected to melting curve analysis and quantification by comparing fluorescence intensity in the linear amplification range. Relative expression of selected genes is quantified by an established method (Livak and Schmittgen, Methods 25:402-408, 2001) using the housekeeping gene, GAPDH, for normalization and a total spinal cord sample as a reference.

In situ hybridization. Most cDNA fragments corresponding to target sequences on the Affymetrix array can be purchased from distributors of the Integrated Molecular Analysis of Genomes and their Expression (I.M.A.G.E) consortium (http://image.llnl.gov/image/html/idistributors.shtml) and the Institute of Physical and Chemical Research (RIKEN) (http://www.dnaform.co.jp/English/Dnaform_E.htm). We clone cDNAs not in the I.M.A.G.E or RIKEN database by PCR with primers designed to recognize the target sequence. For in situ hybridization, we use the following protocol (Chatterton et al., Nature 415:793-798, 2002). Briefly, RNA probes are synthesized from selected cDNA regions near the 3′ end showing the least similarity to other mouse cDNAs based on a Blast search of the National Center for Biotechnology Information (NCBI) website. cDNA fragments are subcloned by PCR into the pCR-TOPO vector using a PCR cloning kit (Invitrogen, Carlsbad, Calif.). The orientation of these inserts allows us to perform in vitro transcription with T₃ and T₇ polymerases producing antisense and sense RNA, respectively. RNA probes are labeled by a non-radioisotopic (DIG, digoxigenin) method.

Mice are transcardially perfused with saline followed by 4% paraformaldehyde in PBS (pH 7.4) under isoflurane anesthesia. The brain is dissected and cryo-protected in 25% sucrose/PBS overnight at 4° C. Sections of 20 μm are prepared using a cryostat and immediately mounted onto poly-L-lysine coated slides. Sections are treated with a solution containing 0.0005% proteinase K to reduce non-specific probe binding and incubated with an RNA probe (100 ng/ml) overnight at 55-63° C. Control hybridization is conducted using either sense probes or no probe on normal sections and antisense probes on sections pretreated with RNase. After hybridization, sections are treated with RNase A at 37° C. for 30 min to further decrease non-specific hybridization. Digoxigenin-labeled sections are incubated with sheep anti-DIG-alkaline phosphatase antibody (Fab fragments), followed by a standard staining method using nitro blue tetrazolium chloride (18.75 g/ml) and 5-bromo-4-chloro-3-indolyl phosphate (9.4 mg/ml) (Roche, Mannheim, Germany) to detect alkaline phosphatase activity. Images are collected using Adobe Photoshop on a Macintosh computer and a microscope equipped with a digital camera.

Immunohistochemistry. ISG15 and UBP43 antibodies (Malakhov et al., J. Biol. Chem. 277:9976-9981, 2002; Malakhova et al., Genes Dev. 17:455-460, 2003) are used for immunohistochemistry studies of ISG15. Rabbit polyclonal antibodies are available commercially for additional candidate biomarker s including IF127 and OAS1A (abcom, Cambridge, Mass.). Antibodies for the neuronal marker MAP 2A, the astrocyte marker GFAP, and the microglia marker Iba-I, are also commercially available.

For immunohistochemistry, we employ a protocol similar to that reported in our published papers (Chatterton et al., Nature 415:793-798, 2002; Wang and Zhang, Eur. J. Neurosci. 22:2376-2380, 2005; Xing, et al., Eur. J. Neurosci. 24: 2987-2992, 2006). Briefly, mice are anesthetized with isofluorane and then transcardially perfused with saline followed by perfusion with 4% paraformaldehyde in 0.1 M phosphate buffered saline (PBS) (pH 7.4). Tissues are dissected and cryoprotected in 25% sucrose/PBS overnight at 4° C. Brain tissues are blocked into Optimal Cutting Temperature (O.C.T.) compound (Sakura Finetec U.S.A. Inc. Torrance, Calif.) and frozen on a bed of methylbutane and dry ice. Frozen tissue is sectioned (12 μm) on a cryostat and mounted onto slides. Sections are air-dried at least one hour before staining. Slides with frozen tissue sections are rinsed twice with PBS+0.1% Triton-X-100 (PBST) for 10 min each and then blocked with 5% normal goat serum in PBST+1% BSA for 1 hr at room temperature. Slides will then be incubated with primary antibody (1:500 dilution) in PBST+1% BSA overnight at 4° C. Slides are then rinsed three times in PBST+0.25% BSA for 5 min each, followed by incubation with a biotinylated secondary antibody (1:500 dilution) (Vector, Burlingame, Calif.) in PBST+0.25% BSA for 1 hr at room temperature. Sections are washed to remove unbound secondary antibody and incubated with freshly prepared AB buffer (ABC kit, Vector, Burlingame, Calif.) for 1 hr. Sections will then be incubated with a solution containing 0.5% diaminobenzidine (DAB) for 2 to 30 min, depending on signal intensity and signal/background ratio. Finally, tissue sections are dehydrated through progressive ethanol solutions (70%, 90%, and 100% ethanol) into xylene, mounted onto slides, cleared, and coverslipped for examination.

Western blot analysis. We conduct Western blot analysis for marker expression using a previously established methods in our laboratory (Chatterton et al., Nature 415:793-798, 2002; Xing, et al., Eur. J. Neurosci. 24: 2987-2992, 2006). Briefly, pre-symptomatic SOD1 (G93A) mice or littermate controls are decapitated under deep anesthesia with isoflurane. The ventral grey matter from the lumbar region of the spinal cords is isolated and homogenized in 50 mM Tris-acetate buffer (pH 7.4) containing 150 mM NaCl, 1% Nonidet p-40, and 1×Halt Protease Inhibitor Cocktail (Pierce, Rockford, Ill., USA). Supernatants are obtained from lysates by centrifugation (16,000×g at 4° C. for 5 min), and the total protein concentration in each sample is determined spectrophotometrically. An aliquot of each sample containing 30 mg protein/10 mL is denatured in gel-loading buffer and proteins are separated on sodium dodecyl sulfate-polyacrylamide (SDS-PAGE) gels. Following electrophoresis, proteins are transferred to nitrocellulose membranes, and membranes are incubated with a primary antibody for the biomarker. Subsequently, the membrane is incubated with the appropriate secondary antibody conjugated with peroxidase. Finally, the membrane is processed with the enhanced chemiluminescence (ECL) plus detection system (Amersham, Piscataway, N.J., USA) and exposed to Kodak Biomax film.

Histology. We conduct histological examination for the general morphology of tissue samples in parallel with biomarker analysis. For histological evaluation, the number of motor neurons in layers VIII and IX of the lumbar spinal cord is compared at appropriate age intervals, as identified by HB9 staining (Sigma, St. Louis, Mo.) or staining with SMI-32 antibody (Sternberger Monoclonals), and counted using the stereological method [9]. The overall appearance of motor neurons is also examined by conventional methods used to analyze SOD1 mutant mice, including hematoxylin/eosin and cresyl violet staining of frozen sections.

Interpretation of data We investigate changes in SOD1 mutant mice during disease progression to determine the usefulness if products of these genes as biomarkers for the disease. By gene profiling we have identified a group of interferon-stimulated genes up-regulated in pre-symptomatic stages for use as disease biomarkers. Determining the temporal and spatial expression patterns and levels of these genes by Q-PCR, in situ hybridization, immunohistochemistry, and Western blot analysis confirms our gene profiling results but also assists in the evaluation of their usefulness as disease biomarkers. If expression of a biomarker indicates disease progression, its spatial expression pattern should match that of cells affected in mutant mice. The temporal expression pattern of a biomarker should also match the disease time course in mutant mice. Both cases have been confirmed (Examples 1 and 2 above). Using this highly sensitive strategy, we accurately detect expression changes of candidate biomarkers during disease progression and identify ideal biomarkers that show statistically significant changes. These studies also are useful for identifying biomarkers in different cell types, including neuronal and glial cells. Biomarkers suitable for detecting motor neuron disease are also useful for other neurodegenerative diseases.

As disease affects only small region of the spinal cord, harvesting total spinal cord as a sample may reduce the sensitivity of the detection method and cause variation in the data. Therefore, it is possible to employ an alternative approach using a different sampling method, which is more accurate but tedious. In such a case frozen sections of spinal cord are made and laser capture microdissection (LCM) is used to isolate small samples from the ventrolateral quadrant where affected motor neurons and neighboring cells reside.

### EXAMPLE 4

### Use of Biomarkers Identified in SOD1 Mice as Biomarkers of a Variety of Neurodegenerative Diseases

Biomarkers identified in SOD1 mutant mice are also expected to be associated with neuronal injury and, therefore, are useful as biomarkers of general neuronal injury in other diseases. The expression of these biomarkers is confirmed in mouse models of prevailing neurodegenerative diseases including Alzheimer's disease (AD), Parkinson disease (AD), Huntington's disease (HD), and stroke. Such animal models are available and well characterized in terms of disease progression and pathological changes in the brain. These models also have diversified types (acute and chronic) and sites of injury and, therefore, are suitable for testing general biomarkers. The use of general biomarkers for these diseases has many advantages over disease type-specific biomarkers. Also equally important, the use of general biomarkers has a wider application and is thus more cost-effective.

To evaluate the expression of biomarkers in other neurodegenerative diseases, we select commonly used and well-characterized mouse models for AD, PD, HD, and stroke. In addition, we select chronic AD and HD models, in which symptoms are manifested for weeks or months after birth, and acute PD and stroke models, in which symptoms are manifested within days of insult. Such analyses enable us to determine the expression of biomarkers at disease onset, progression, and in recovery phases when applicable. We first analyze expression of ISG15, a biomarker confirmed in our preliminary studies, in these mouse models by in situ hybridization and immunohistochemistry. We then analyze additional biomarkers. After confirming expression of biomarkers, we take samples of brain tissue in which a particular biomarker is expressed for quantitative analysis using Q-PCR and Western blot analysis in order to determine the sensitivity, specificity, and robustness of each biomarker in each disease state. We then confirm each biomarker's reproducibility and its range of coverage in all disease types.

Animal model for Alzheimer's Disease (AD). For an animal model of AD use a double transgenic mouse line containing an exon 9 deletion mutant of presenilin 1 (PSEN1dE9) and a chimeric amyloid precurser protein containing the Swedish mutation K595N/M596L (APPswe) (Jankowsky et al., Hum. Mol. Genet. 13:159-170, 2004; Jankowsky et al., Biomol. Eng. 17:157-165, 2001). Both transgenes are driven by the mouse prion promoter, were co-integrated, and co-segregate as a single locus. Histological examination of mouse brain tissue at six months of age reveals amyloid deposits in neocortex and hippocampus resembling those observed in the brains of AD patients. The number of amyloid deposits increases dramatically after nine months. A specific increase of Aβ42 levels due to PSEN1dE9 activity likely accounts for accelerated amyloid deposition. Thus, these mice are useful to study expression of biomarkers relating to amyloid deposition, a process implicated in cell death.

We use a double transgenic mouse line [Strain name: Tg(APPswe,PSEN1dE9)85Dbo/0] (Jackson Laboratory, Bar Harbor, Me.). Hemizygotes are crossed with wild-type mice in order to compare mutant and wild-type mice from the same litter. We harvest brain samples at 3, 6, 9, 12 and 18 months of age. Analysis is focused on the neocortex and hippocampus.

Animal model for Huntington's Disease (HD). For an HD model we use the R6/2 mouse line, the most widely used transgenic mouse model of HD (Li et al., NeuroRx 2:447-464, 2005). This mouse line uses human HD promoter to drive a small N-terminal fragment (exon 1) of human huntingtin containing approximately 150 CAG repeats. Among all HD transgenic models, the R6/2 model develops symptoms most rapidly: mice become hyperactive at 3 weeks of age and then gradually hypoactive around seven weeks, severely impaired by 8-12 weeks, and dead at 13-16 weeks. Little cell death is reported in the R6/2 brain, but some is seen in the striatum and hypothalamus. Nonetheless, total brain volume is reduced by 20% at 12 weeks of age, potentially reflecting atrophy of neurons and a decrease in the neuropil. The relatively quick disease onset and clear defined survival time makes R6/2 model a good choice for biomarker studies.

The R6/2 mouse line is used (strain name: B6CBA-Tg(HDexon1)62Gpb/2J, Jackson Laboratory, Bar Harbor, Me.). Hemizygote males are bred with C57/bl females to generate offspring composed of hemizygote mutants and wild-type controls. We will take brain samples from 2-, 4-, 8- and 12-week old mice, focusing particularly in the area of the striatum and hypothalamus.

Animal model for Parkinson's Disease (PD). We employ a PD model based on MPTP administration to C57/black6 mice (Jackson-Lewis et al., Neurodegeneration 4:257-269, 1995), a treatment that induces neuronal degeneration in the substantia nigra pars compacta (SNpc) (Q30%) and the ventral tegmental area (VTA) (˜8%). The active phase of degeneration begins at 12 h post-injection and continues for up to 4 days. Dying neurons show shrunken eosinophilic cytoplasm and shrunken, darkly stained nuclei. However, during the four-day period, MPTP causes a greater decrease in tyrosine hydroxylase (TH)-positive neurons, which are dopaminergic (DA), than in Nissl-stained neurons, suggesting that some injured neurons lose only TH activity without undergoing degeneration. After four days there is no further loss of dopaminergic neurons. In contrast to other neurodegenerative disease models used here, in this model there is no evidence of an inflammatory reaction or increased numbers of glial cells in the SNpc and VTA.

We use eight-week-old C57/black6 mice (22-25 g). On the first day of the study, mice receive four intraperitoneal (i.p.) injections of control saline or MPTP-HCl (20 mg/kg free base; Sigma St. Louis, Mo.) in saline at 2 h intervals. We take brain samples at 6 and 12 hours, and then at 1, 3, 10, 20, and 30 days post-injection. Analysis will focus on the SNpc and VTA.

Animal model for stroke. We use a stroke model employing 30 min of bilateral common carotid artery (BCCA) occlusion, which is particularly effective in C57/black6 mouse strain (Fujii et al., Stroke 28:1805-1810, discussion at 1811, 1997). This model creates transient forebrain ischemia damaging the hippocampus and, to a lesser extent, the striatum, cerebral cortex, and thalamus. 72 hours after reperfusion, neuronal loss in these areas can reach 30-70%. The most significant injury can be observed in the CA1 region of the hippocampus, where shrunken and dark pyknotic neurons are concentrated. Small to medium-sized striatal neurons in the neostriatum are also affected, but the globus pallidus is resistant to ischemic injury. In addition, ischemic changes are limited to layers 2 and 3 of the cerebral cortex but are consistent in different thalamic nuclei. Mortality is not seen, and the injury pattern is consistent in this model.

We use wild-type C57/black6 mice (Jackson Laboratory, Bar Harbor, Me.) and employ a simple surgical procedure. Briefly, under anesthesia induced by 2% halothane and maintained by 1% halothane, the common carotid arteries are exposed after a ventral midline cervical incision and ligated with 6-0 silk sutures. During the 30 min ischemia period, anesthesia is terminated and animals monitored for signs of consciousness. Ligatures are then removed to reperfuse animals while again under brief anesthesia by 1% halothane. The incision is closed when blood flow returns. We take brain samples at 1, 3, 5, 10, and 20 days after ischemia for analysis and focus primarily on the CA1 region of the hippocampus.

Expression of biomarkers. We employ methods similar to those described above for general histology, in situ hybridization, Q-PCR, immunohistochemistry and Western analysis. Brain samples from each animal model are taken according to respective disease type, and coronal or sagittal sections are made to best view affected areas. As we are interested in pre-symptomatic stages of chronic disease models, we refine sampling time points after initial testing to determine when biomarker expression is initiated. We also refine sampling time points at late stages of acute disease models to determine whether biomarkers are expressed in potentially recovery phases of diseases.

Parallel samples are collected to examine both biomarker expression and the general health of tissues. Besides histological examination, we also employ disease-specific methods to reveal pathological changes in a particular disease model. For the AD model, we use the commonly used Congo Red method to stain amyloid plaques following a previously established protocol (Jankowsky et al., Biomol. Eng. 17:157-165, 2001). Briefly, 10 μm brain paraffin sections are mounted onto slides, counterstained in hematoxylin, and immersed in congo red solution (1% Congo Red, 4% NaCl, 80% ethanol) for 30 min. Slides are moved into aniline blue (7.5 g aniline blue, 5 ml glacial acetic acid, and 250 ml distilled water) and then dehydrated in ethanol and xylene. For the PD model, we examine the loss of TH in the striatum and SN by immunostaining (Jackson-Lewis et al., Neurodegeneration 4:257-269, 1995), as this is a more sensitive method to detect cell injury in this model than examining cell death. We use a rabbit polyclonal anti-TH antibody (abcom, Cambridge. MA) and follow our established protocol. As there is no convenient disease-specific marker for the HD and stroke models, we monitor gliosis, which occurs in both models during disease progression, by immunostaining with an astrocyte marker GFAP and a microglia marker Iba-1.

We determine similarities and differences in biomarker expression in various disease models. The use of chronic and acute disease models reveals temporal patterns of biomarkers. The spatial pattern of a particular biomarker will identifies the origin of the disease and the scope of brain injury.

### REFERENCES

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A method of detecting neurodegeneration in an individual, comprising providing a sample from the individual comprising a neural cell; detecting levels of a biomarker polypeptide in the sample; and comparing the levels of the biomarker polypeptide in the sample to levels of the biomarker polypeptide in a control sample; wherein expression of the biomarker polypeptide has a two-fold or greater difference in expression in the spinal cord of 60- and/or 100-day-old SOD1 mutant mice compared with expression in the spinal cord of wild-type littermates.

2. The method of claim 1 wherein the biomarker polypeptide is selected from the group consisting of Usp18, Ifi202, Iigp1, Ifi27, Ifit3, Oas12, Ifit1, Rsad2, Ifi44, Isg15, Cxcl10, Gbp2, Socs3, Irf8, Oas1a, Irgm, B2m, Psmb8, Igtp, Isgf3g, Ifitm3, Stat1, Ifih1, Iigp2, Ifit2, Samhd1, and Clec7a polypeptides.

3. The method of claim 1 wherein the biomarker polypeptide is an Isg15 or Usp18 polypeptide.

4. The method of claim 1 wherein the neurodegeneration is a result of a disease or injury selected from the group consisting of Alzheimer's disease, Parkinson's disease, amyotrophic lateral sclerosis, Huntington's disease, encephalitis and dementia resulting from infection with Human Immunodeficiency Virus, brain ischemia, stroke, trauma, viral infection and prion infection.

5. The method of claim 1 comprising contacting the sample with an antibody that binds selectively to the biomarker polypeptide, and detecting binding of the antibody to the biomarker polypeptide.

6. The method of claim 5 wherein the antibody is a monoclonal antibody.

7. The method of claim 5 comprising performing an ELISA assay.

8. The method of claim 5 comprising performing a bio-barcode assay.

9. An automated method of claim 1.

10. A method of detecting neurodegeneration in an individual, comprising providing a sample from the individual comprising a neural cell; detecting levels of a biomarker polynucleotide in the sample; and comparing the levels of the biomarker polynucleotide in the sample to levels of the biomarker polynucleotide in a control sample; wherein expression of the biomarker polynucleotide has a two-fold or greater difference in gene expression in the spinal cord of 60- and/or 100-day-old SOD1 mutant mice compared with expression in the spinal cord of wild-type littermates.

11. The method of claim 10 wherein the biomarker polynucleotide is selected from the group consisting of Usp18, Ifi202, Iigp1, Ifi27, Ifit3, Oas12, Ifit1, Rsad2, Ifi44, Isg15, Cxcl10, Gbp2, Socs3, Irf8, Oas1a, Irgm, B2m, Psmb8, Igtp, Isgf3g, Ifitm3, Stat1, Ifih1, Iigp2, Ifit2, Samhd1, and Clec7a polynucleotides.

12. The method of claim 10 wherein the biomarker polynucleotide is an Isg15 or Usp18 polynucleotide.

13. The method of claim 10 wherein the neurodegeneration is a result of a disease or injury selected from the group consisting of Alzheimer's disease, Parkinson's disease, amyotrophic lateral sclerosis, Huntington's disease, encephalitis and dementia resulting from infection with Human Immunodeficiency Virus, brain ischemia, stroke, trauma, viral infection and prion infection.

14. The method of claim 10 comprising contacting the sample with a first primer that comprises the polynucleotide sequence that hybridizes selectively to the biomarker polynucleotide and a second primer comprising a polynucleotide sequence that hybridizes to the biomarker polynucleotide, performing an amplification reaction, and quantitating an amplification product of the biomarker polynucleotide in the sample.

15. The method of claim 11 wherein the amplification reaction is a PCR reaction.

16. The method of claim 12 wherein the amplification reaction is a quantitative PCR reaction.

17. The method of claim 11 comprising performing a bio-barcode assay.

18. An automated method of claim 10.

19. A method of assessing the effectiveness of a course of treatment for an individual suffering from a neurodegenerative disease or neural cell damage, the method comprising (a) measuring a first level of a biomarker polypeptide in a sample from the individual at a first time point during the course of treatment, (b) measuring a second level of the biomarker polypeptide in a sample from the individual at a second time point during the course of treatment, and (c) comparing the measurements of the biomarker polypeptide at the first timepoint and the second timepoint; wherein expression of the biomarker polypeptide has a two-fold or greater difference in expression in the spinal cord of 60- and/or 100-day-old SOD1 mutant mice compared with expression in the spinal cord of wild-type littermates.

20. An automated method of claim 19.

21. A method of assessing the effectiveness of a course of treatment for an individual suffering from a neurodegenerative disease or neural cell damage, the method comprising (a) measuring a first level of a biomarker polynucleotide in a sample from the individual at a first time point during the course of treatment, (b) measuring a second level of the biomarker polynucleotide in a sample from the individual at a second time point during the course of treatment, and (c) comparing the measurements of the biomarker polynucleotide at the first timepoint and the second timepoint; wherein expression of the biomarker polynucleotide has a two-fold or greater difference in expression in the spinal cord of 60- and/or 100-day-old SOD1 mutant mice compared with expression in the spinal cord of wild-type littermates.

22. An automated method of claim 21.

23. A method of assessing the progression of a neurodegenerative disease in an individual suffering from the neurodegenerative disease or neural cell damage, the method comprising (a) measuring a first level of a biomarker polypeptide in a sample from the individual at a first time point during the course of treatment, (b) measuring a second level of the biomarker polypeptide in a sample from the individual at a second time point during the course of treatment, and (c) comparing the measurements of the biomarker polypeptide at the first timepoint and the second timepoint; wherein expression of the biomarker polypeptide has a two-fold or greater difference in expression in the spinal cord of 60- and/or 100-day-old SOD1 mutant mice compared with expression in the spinal cord of wild-type littermates.

24. An automated method of claim 23.

25. A method of assessing the progression of a neurodegenerative disease or neural cell damage in an individual suffering from the neurodegenerative disease, the method comprising (a) measuring a first level of a biomarker polynucleotide in a sample from the individual at a first time point during the course of treatment, (b) measuring a second level of the biomarker polynucleotide in a sample from the individual at a second time point during the course of treatment, and (c) comparing the measurements of the biomarker polynucleotide at the first timepoint and the second timepoint; wherein expression of the biomarker polynucleotide has a two-fold or greater difference in expression in the spinal cord of 60- and/or 100-day-old SOD1 mutant mice compared with expression in the spinal cord of wild-type littermates.

26. An automated method of claim 25.

27. A method for identifying a molecule for diagnosing neurodegeneration or neural cell damage in an individual, the method comprising: (1) providing a sample from the individual comprising a biomarker polypeptide; (2) contacting the sample with a test molecule; (3) determining whether the test molecule binds to, or is bound by, the biomarker polypeptide; wherein expression of the biomarker polynucleotide has a two-fold or greater difference in expression in the spinal cord of 60- and/or 100-day-old SOD1 mutant mice compared with expression in the spinal cord of wild-type littermates.

28. The method of claim 27 comprising determining whether the test molecule crosses the blood-brain barrier.

29. An automated method of claim 27.


SEQUENCE LISTING (20 sequences)

SEQ ID NO: 1 - DNA, 728 bp, Mus musculus
gagctgggggggggcaggggggggagagtcgatccagtctctgactgtgagagcaagcagccagaagcagactccttaattccaggggacctagagctagagcctgcagcaatggcctgggacctaaaggtgaagatgctggggggtaacgatttcctggtgtccgtgactaactccatgacggtgtcagaactgaagaagcagattgcccagaagattggtgtgccggctttccagcagcgcctggcccaccaaactgcagtgctccaggacggtcttaccctttccagtctgggtctgggtcccagcagcacagtgatgctagtggtacagaactgcagcgagcctctgagcatcctggtgaggaacgaaaggggccacagcaacatctatgaggtctttctgacgcagactgtagacacgcttaagaagaaggtgtccagcggaacaagtcacgaagaccagttctggctgagcttcgagggaaggcccatggaggacaaggagctgctgggggagtatggcctaaagccccagtgcacagtgatcaagcatttgcgcctgaggggtgggggaggggaccagtgtgcctaaggcccccatccagagccagtgttggttataaaagatgatgggggggggggtatggaggacttttgggatagcattggaaatgtaaacgaggaaaatacctaattagaaaaaatatcaaaaaaaaaaaaaagctc

SEQ ID NO: 2 - protein, 160 aa, Mus musculus
MAWDLKVKMLGGNDFLVSVTNSMTVSELKKQIAQKIGVPAFQQRLAHQTAVLQDGLTLSSLGLGPSSTVMLVVQNCSEPLSILVRNERGHSNIYEVFLTQTVDTLKKKVSSGTSHEDQFWLSFEGRPMEDKELLGEYGLKPQCTVIKHLRLRGGGGDQCA

SEQ ID NO: 3 - DNA, 24 bp, Artificial Sequence
  Description of Artificial Sequence Synthetic primer
tgtagaccaaaagttccagacaga

SEQ ID NO: 4 - DNA, 20 bp, Artificial Sequence
  Description of Artificial Sequence Synthetic primer
gataaaggcatctcgcttgg

SEQ ID NO: 5 - DNA, 20 bp, Artificial Sequence
  Description of Artificial Sequence Synthetic primer
tagccacactccaatcagca

SEQ ID NO: 6 - DNA, 19 bp, Artificial Sequence
  Description of Artificial Sequence Synthetic primer
agagcaaggctccaacagc

SEQ ID NO: 7 - DNA, 27 bp, Artificial Sequence
  Description of Artificial Sequence Synthetic primer
ctgattacaaaagaagacatgacagac

SEQ ID NO: 8 - DNA, 20 bp, Artificial Sequence
  Description of Artificial Sequence Synthetic primer
aggcaaaaccaaagactcca

SEQ ID NO: 9 - DNA, 20 bp, Artificial Sequence
  Description of Artificial Sequence Synthetic primer
ggacaaggtggagaaggtgt

SEQ ID NO: 10 - DNA, 20 bp, Artificial Sequence
  Description of Artificial Sequence Synthetic primer
tcctcacagtccatctcagc

SEQ ID NO: 11 - DNA, 20 bp, Artificial Sequence
  Description of Artificial Sequence Synthetic primer
ggaatgcccagaacttgaat

SEQ ID NO: 12 - DNA, 20 bp, Artificial Sequence
  Description of Artificial Sequence Synthetic primer
gctcgttcatttcttccaca

SEQ ID NO: 13 - DNA, 21 bp, Artificial Sequence
  Description of Artificial Sequence Synthetic primer
gaacaagtccacgaagaccag

SEQ ID NO: 14 - DNA, 19 bp, Artificial Sequence
  Description of Artificial Sequence Synthetic primer
gcagctccttgtcctccat

SEQ ID NO: 15 - DNA, 18 bp, Artificial Sequence
  Description of Artificial Sequence Synthetic primer
gctgccagcctttgatgt

SEQ ID NO: 16 - DNA, 20 bp, Artificial Sequence
  Description of Artificial Sequence Synthetic primer
tggcatagattctgggatca

SEQ ID NO: 17 - DNA, 20 bp, Artificial Sequence
  Description of Artificial Sequence Synthetic primer
ggcagtgcttaggtgacaga

SEQ ID NO: 18 - DNA, 20 bp, Artificial Sequence
  Description of Artificial Sequence Synthetic primer
gagagatcccatgaaccgat

SEQ ID NO: 19 - DNA, 20 bp, Artificial Sequence
  Description of Artificial Sequence Synthetic primer
ggcattgctctcaatgacaa

SEQ ID NO: 20 - DNA, 20 bp, Artificial Sequence
  Description of Artificial Sequence Synthetic primer
tgtgagggagatgctcagtg